(19) United States
(12) Patent Application Publication
(10) Pub. No.: US 2022/0098742 A1
(43) Pub. Date: Mar. 31, 2022

(54) CATALYST MATERIAL FOR ENHANCING HYDROGEN AND OXYGEN PRODUCTION AND SYNTHESIZING METHODS OF SAME

(71) Applicant: BOARD OF TRUSTEES OF THE UNIVERSITY OF ARKANSAS, Little Rock, AR (US)

(72) Inventor: Wei Zhao, Little Rock, AR (US)

(21) Appl. No.: 17/487,072

(22) Filed: Sep. 28, 2021

Related U.S. Application Data
(60) Provisional application No. 63/084,079, filed on Sep. 28, 2020.

(51) Int. Cl. C25B 11/065; C12N 1/12; C12P 3/00; C25B 1/04; C25B 11/054; C25B 11/077

(52) U.S. Cl. CPC C25B 11/065; C12N 1/12; C25B 11/077; C25B 1/04; C25B 11/054; C12P 3/00

(57) ABSTRACT

A catalyst material for enhancing hydrogen and oxygen production includes algae-derived carbon scaffolds; and catalyst components coupled to the algae-derived carbon scaffolds. The catalyst material has excellent oxygen evolution reaction (OER) performance superior to that of a benchmark OER catalyst Ir/C.

### CROSS-REFERENCE TO RELATED PATENT APPLICATION

This application claims priority to and the benefit of U.S. Provisional Application No. 63/084,079, filed Sep. 28, 2020, which is incorporated herein in its entirety by reference.

### FIELD OF THE INVENTION

The present invention relates generally to materials, and more particularly to a catalyst material for enhancing hydrogen and oxygen production and synthesizing methods of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Earth-abundant microalgae and cyanobacteria, as photosynthetic microorganisms, have emerged as an attractive new high-potential farmable bioresource such as biofuels and new raw materials for green chemistry. As a renewable and sustainable source, their main advantages are solar production with higher surface productivities than plants, and a carbon-neutral operation by simultaneously consuming carbon dioxide.

Carbon-based scaffolds have been used to generate highly efficient, low-cost, earth-abundant water-splitting nanocatalysts. The scaffolds provide crucial morphology controls for growing size-controllable nanocatalysts less than 10 nm, with optimal sizes of ˜2-5 nm. To take the advantage of the carbon-based scaffolds, biotemplating is an effective strategy to obtain morphology-controllable materials with structural specificity, complexity, and corresponding unique functions. Biological templates such as viruses, bacteria, algae, and other microorganisms have a plethora of shapes that could be of interest for a broad range of technological applications. These templates usually exhibit complex morphologies containing turns, coils, angles, and pores, and their size varies from tens of micrometers for algae to nanometers for viruses. In addition, they can be organized into three-dimensional (3D) hierarchical structures via bioconjugation techniques to create porous films or arrays. A few recent studies have explored microalgae and cyanobacteria-based biotemplates to synthesize hollow porous MnO/C microspheres, biogenic carbon-doped titania, hollow and solid magnetic silica microspheres, rattle-type multiple magnetite cores microspheres with porous biopolymer shell, and tin-decorated carbon Sn@C composites. However, none of these studies used cell-derived carbon nanostructures for synthesizing water-splitting nanocatalysts.

Therefore, a heretofore unaddressed need exists in the art to address the aforementioned deficiencies and inadequacies.

### SUMMARY OF THE INVENTION

In one aspect, the invention relates to a catalyst material for enhancing hydrogen and oxygen production, comprising algae-derived carbon scaffolds; and catalyst components coupled to the algae-derived carbon scaffolds.

In one embodiment, the algae-derived carbon scaffolds comprise algae-derived carbonized cells (cCells).

In one embodiment, the algae-derived carbon scaffolds are formed by carbonization of algae cells.

In one embodiment, the algae cells comprise Tetraselmis cells, Nannochloropsis gaditana, Nannochloropsis oculate, or the likes.

In one embodiment, the algae-derived carbon scaffolds comprise three-dimensional (3D) reduced graphene oxide (RGO) scaffolds.

In one embodiment, the algae-derived carbon scaffolds comprise about 77 atomic % of C and about 14 atomic % of O.

In one embodiment, the algae-derived carbon scaffolds contain C═C bonds, hydroxyl C—OH bonds, and ester C(═O)O bonds, wherein the C═C bonds are dominant bonds.

In one embodiment, the catalyst components comprise efficient oxygen evolution reaction (OER) and hydrogen evolution reaction (HER) catalysts with earth-abundant materials, transition metal oxides/layer-double-hydroxides including NiFe oxide (NiFeOₓ), cobalt phosphate, perovskite oxides, and transition metal dichalcogenides including MoS₂.

In one embodiment, the NiFe oxide has a molar ratio of Ni:Fe:O=6.7:6.1:26, with a formula of Ni_{1.1}FeO_{4.3}.

In one embodiment, the catalyst material has a molar ratio of C:O:Ni:Fe≈49:35:6.7:6.1.

In one embodiment, the catalyst material has a molar ratio of cCells to NiFe oxide, (C:O)_{cCell}:(Ni:Fe:O)_{NiFeOx}=49:9:6.7:6.1:26.

In one embodiment, the catalyst material has about 39 wt. % of cCells and about 61 wt. % of NiFe oxide.

In one embodiment, the catalyst material has Ni species mostly in the +2 oxidation state (NiOₓH_{y}) with Ni 2p_{3/2} binding energies close to 856 eV, and Fe species mostly in the +3 oxidation state (Fe₂O₃/FeOOH) with Fe 2p_{3/2} binding energies close to 711 eV.

In one embodiment, the catalyst material has oxygen evolution reaction (OER) performance superior to that of a benchmark OER catalyst Ir/C.

In another aspect, the invention relates to an electrochemical device for hydrogen and oxygen production, comprising at least one electrode comprising the catalyst material as disclosed above.

In yet another aspect, the invention relates to a method for synthesizing a catalyst material for enhancing hydrogen and oxygen production, comprising filling algal cells with Ni²⁺ ions and Fe³⁺ ions to form a Ni²⁺/Fe³⁺@Cell composite comprising the Ni²⁺ and Fe³⁺ ions and the algal cells; mixing NH₃.H₂O with the Ni²⁺/Fe³⁺@Cell composite to form a NiFe(OH)ₓ@Cell composite comprising NiFe(OH)ₓ and the algal cells; mixing tetramethoxysilane (TMOS) with the NiFe(OH)ₓ@Cell composite to form a NiFe(OH)ₓ@Cell-SiO₂ composite comprising NiFe(OH)ₓ, the algal cells and SiO₂; pyrolyzing the NiFe(OH)ₓ@Cell-SiO₂ composite at a temperature in a range of about 500-900° C. to form a NiFeOₓ@cCell-silica composite comprising NiFe(OH)ₓ, algae-derived carbonized cells (cCell) and silica; and removing the silica from the NiFeOₓ@cCell-silica composite to obtain the catalyst material.

In one embodiment, said filling the algal cells with the Ni²⁺ ions and the Fe³⁺ ions to form the Ni²⁺/Fe³⁺@Cell composite comprises adding the algae cells into a first solution containing the Ni²⁺ ions and the Fe³⁺ ions to form a first mixture thereof, and shaking the first mixture for a period of time at room temperature, then centrifuging and washing the first mixture using DI water until the upper solution is colorless and no precipitates are formed when a NaOH solution is added, and collecting solids as the Ni²⁺/Fe³⁺@Cell composite.

In one embodiment, the first solution has a mole ratio of Ni²⁺:Fe³⁺=3:1.

In one embodiment, said mixing the NH₃.H₂O with the Ni²⁺/Fe³⁺@Cell composite to form the NiFe(OH)ₓ@Cell composite comprises mixing the Ni²⁺/Fe³⁺@Cell composite with a second solution containing DI water, ethanol and concentrated NH₃.H₂O to form a second mixture; and shaking the second mixture for a second period of time, then centrifuging and washing the second mixture until a final pH˜8.93 in the upper solution, and collecting solids as the NiFe(OH)ₓ@Cell composite.

In one embodiment, said mixing TMOS with the NiFe(OH)ₓ@Cell composite to form the NiFe(OH)ₓ@Cell-SiO₂ composite comprises mixing the NiFe(OH)ₓ@Cell composite with a third solution containing DI water, ethanol and TMOS to form a third mixture; and shaking the third mixture to form a homogeneous gel and drying homogeneous gel to obtain the NiFeOₓ@Cell-SiO₂ composite.

In one embodiment, said pyrolyzing is performed in N₂.

In one embodiment, said removing the silica from the NiFeOₓ@cCell-silica composite comprises adding the NiFeOₓ@cCell-SiO₂ composite into a fourth solution containing NaOH to form a fourth mixture; heating the fourth mixture to a temperature in a range of about 60-120° C. on a hot plate and keeping the fourth mixture for about 4 hours at the temperature with mild stirring, and then cooling the fourth mixture down to room temperature; and centrifuging, washing with DI water, and dry the fourth mixture to obtain the NiFeOₓ@cCell.

In yet another aspect, the invention relates to method for synthesizing a catalyst material for enhancing hydrogen and oxygen production, comprising preparing a cell suspension comprising algal cells; mixing tetramethoxysilane (TMOS) with a cell suspension to form a Cell-SiO₂ composite; pyrolyzing the Cell-SiO₂ composite at a temperature in a range of about 500-900° C. to form a carbonized Cell-SiO₂ (cCell-SiO₂) composite; removing silica from the cCell-SiO₂ composite to obtain the carbonized cells (cCells); loading precursors into the cCells to form a precursor-cCell mixture; and performing hydrothermal reaction on the precursor-cCell mixture for 2-4 hours at a temperature in a range of about 100-250° C. to obtain the catalyst material.

In one embodiment, the algal cells comprise tetraselmis cells.

In one embodiment, said mixing TMOS with the cell suspension to form the Cell-SiO₂ composite comprises shaking a mixture of the TMOS with the cell suspension for one day at room temperature to obtain the Cell-SiO₂ composite.

In one embodiment, said pyrolyzing is performed in N₂.

In one embodiment, said removing silica from the cCell-SiO₂ composite comprises heating a mixture of the cCell-SiO₂ composite with a NaOH solution to a temperature in a range of about 60-120° C. for about 2-6 hours on a hot plate, and then cooling the mixture down to room temperature; and centrifuging, washing, and drying the mixture to obtain the carbonized cells (cCells).

In one embodiment, the composition of the cCell comprises 77 atomic % of C and 14 atomic % of O.

In one embodiment, said loading the precursors into the cCells comprises preparing a metal ion mixed solution containing Ni²⁺ and Fe³⁺ ions; and adding the cCells into the metal ion mixed solution to form the precursor-cCell mixture.

In one embodiment, the metal ion mixed solution has a mole ratio of Ni²⁺:Fe³⁺=3:1, and the precursor-cCell mixture has a mole ratio of C:Ni²⁺:Fe³⁺=13:21:7.

In one embodiment, the metal ion mixed solution has a pH of 5.88, the precursor-cCell mixture has a pH of 5.91, and after the hydrothermal reaction, the resulting mixture is centrifuged, and the pH of the upper solution is 5.87.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows schematically a pre-loading reaction route for NiFeOₓ@cCell synthesis, according to embodiments of the invention. Step a): Fill algal cells with Ni²⁺ and Fe³⁺ to form Ni²⁺/Fe³⁺@Cell. Step b): Add NH₃.H₂O to form precursor NiFe(OH)ₓ@Cell. Step c): Add tetramethoxysilane (TMOS), Si(OMe)₄ to NiFe(OH)ₓ@Cell to form NiFe(OH)ₓ@Cell-SiO₂ composite. Step d): Pyrolyze in N₂ at 700° C. to generate NiFeOₓ@cCell-silica composite. Step e): Remove silica using 2 M NaOH.

FIG. 2 shows schematically post-loading reaction routes, according to embodiments of the invention. Step a): Harvest of algal cells. Step b): Cell-silica composite from Si(OMe)₄. Step c): Pyrolysis in N₂ at 700° C. to generate cCell-silica composite. Step d): Silica is removed from the cCell-silica composite using 2 M NaOH to free the cCells. Step e): Embedment of precursors (Ni²⁺ & Fe³⁺ for MOₓ nanomaterials). Step f): Hydrothermal reaction at 180° C.

FIG. 3 shows samples and their optical images, according to embodiments of the invention. Panel a): Tetraselmis cells. Panel b): Cells after reacting with Ni²⁺ and Fe³⁺. Panel c): NiFeOₓ@cCell. Panel d): SEM image of NiFeOₓ@cCell. Panel e): TEM image of a NiFeOₓ@cCell. Panel f): TEM image of the zoom-in area of the red square in panel e) for NiFeOₓ nanoparticles embedded in cCells.

FIG. 4 shows SEM images of a Ni foam and a pre-loading NiFeOₓ@cCell sample, according to embodiments of the invention. Panel a): the SEM image of the Ni foam. Panels b)-d): the SEM images of the pre-loading NiFeOₓ@cCell sample on the Ni foam at different magnifications.

FIG. 5 shows TEM images of Tetraselmis cCells, according to embodiments of the invention. The rod-like objects marked by red arrows in panel b) might be the carbonized flagella of the cells. The inset in panel b) is a broad lateral view of a Tetraselmis cell.

FIG. 6 shows TEM images of cCells in a pre-loading NiFeOx@cCell sample, according to embodiments of the invention. Most of the cells do not contain any particles inside as shown in panel a). Few cells contain particles, distributed both outside shown in panels b)-c) and inside the cells shown in panels d)-f). Image in panel c) is the zoom-in area of the red square in panel b), and images in panels e)-f) are the zoom-in area of the red square in panel d).

FIG. 7 shows a TEM image and EDS spectra of a NiFeOₓ@cCell derived from Tetraselmis and prepared after the hydrothermal reaction step f) at 180° C. for 3 hours, according to embodiments of the invention. Panel a): the TEM image. Panel b): EDS spectra of five spots in panel a) show a nearly homogeneous distribution of Ni/Fe on the cCell.

FIG. 8 shows XPS survey spectrum (panel a) and the composition (panel b) of a cCell sample, according to embodiments of the invention.

FIG. 9 shows Raman spectrum of a cCell sample, according to embodiments of the invention. The line marked with the asterisk near 520 cm⁻¹ was from Si substrate.

FIG. 10 shows XPS spectra of the post-loading NiFeOₓ@cCell sample, according to embodiments of the invention. Panel a): C is XPS spectra of cCell and post-loading NiFeOₓ@cCell samples. Panel b): Ni 2p XPS spectrum of the post-loading NiFeOₓ@cCell sample. Panel c): Fe 2p XPS spectrum of the post-loading NiFeOₓ@cCell sample.

FIG. 11 shows EDS of a pre-loading NiFeOₓ@cCell sample recorded in the area shown in panel f) of FIG. 4. It conforms the Ni and Fe with [Ni]:[Fe]≈3.4:1. Cu is coming from TEM grid. Ca, Na and Cl are possibly coming from the sample.

FIG. 12 shows a general composition survey using EDS for a pre-loading NiFeOx@cCell sample recorded on the large areas of individual cCells containing metal ions, according to embodiments of the invention. It revealed that the Ni and Fe total content was relatively low, about 10 wt % on the NiFe-containing cCells. Since only 25% cCells contained metal ions, roughly, about 2.5 wt % Ni and Fe ions were present in the sample.

FIG. 13 shows EDS spectra of five spots in the TEM image of a cCell loaded with NiFeOₓ nanoparticles prepared via the post-loading method, according to embodiments of the invention. Cu grid was used for TEM imaging.

FIG. 14 shows XPS survey spectra and the chemical composition of a post-loading NiFeOₓ@cCell sample, according to embodiments of the invention. Panels a)-b): Before Ar ion sputtering etching. Panels c)-d): After the first etching. Panels e)-f): After the second etching.

FIG. 15 shows polarization curves and Tafel plots of (1) cCells and (2) pre-loading NiFeOₓ@cCell, according to embodiments of the invention. Panel a) polarization curves and panel b) Tafel plots of (1) cCells and (2) pre-loading NiFeOₓ@cCell on glassy carbon electrodes in 1 M KOH solution. The insert in panel a) is the CV of the pre-loading NiFeOₓ@cCell sample at a scan rate 25 mV/s. Panel c) polarization curves and panel d) Tafel plots of (1) cCells and (2) pre-loading NiFeOₓ@cCell on (0) Ni foam electrodes in 1 M KOH solution. iR-correction was applied.

FIG. 16 shows polarization curves and (b) Tafel plots of post-loading NiFeOₓ@cCell and Ir/C samples, according to embodiments of the invention. Panel a) polarization curves and panel b) Tafel plots of post-loading NiFeOₓ@cCell and Ir/C samples on glassy carbon electrodes in 1 M KOH solution. Panel c) polarization curves and Panel d) Tafel plots of post-loading NiFeOₓ@cCell and Ir/C samples on Ni foam electrodes in 1 M KOH solution. No iR-correction was applied.

FIG. 17 shows OER reaction mechanisms of Ni-based catalysts showing the intermediate steps in the OER (E⁰ versus RHE).

FIG. 18 shows comparison of polarization curves of NiFeOₓ@cCell on Ni foam and Ir/C on Ni foam samples running over two days in 1 M KOH solution.

FIG. 19 shows chronopotentiometric measurement of a post-loading NiFeOₓ@cCell sample on Ni foam electrode of 0.5×0.5 cm² working area in 1 M KOH solution. A three-electrode configuration was used with a current density of 40 mA/cm².

FIG. 20 shows polarization curves of pre-loading and post-loading NiFeOₓ@cCell samples on glassy carbon electrodes in 1 M KOH solution, plotted as Current Density in A/g vs Potential, for comparison of OER activities per NiFeOₓ mass. At 10 A/g NiFeOₓ, the overpotential is 1.66 V for pre-loading sample, and 1.54 V for the post-loading sample, respectively.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Carbon nanostructures are known to serve as a scaffold for growth of efficient water-splitting nanocatalysts. Previous research work for Ni—Fe oxide catalyst synthesis on carbon-based substrates involved carbon precursors in a non-renewable manner. However, how to produce the efficient carbon nanostructures in a carbon-neutral setting is highly challenging.

One of the objectives of this invention is to disclose novel methods for using carbon-neutral algae-based products, i.e., microalgae or cyanobacteria-derived, low-cost, environmentally friendly, highly efficient water-splitting nanocatalysts for hydrogen and oxygen production. The novel methods utilize algal cells as both a renewable and sustainable carbon source and a biotemplate to synthesize low-cost oxygen evolution reaction (OER) NiFe oxide nanocatalysts for highly efficient hydrogen and oxygen production. Using Tetraselmis as an algal example, the nanocatalysts were grown on algae-derived carbonized cells (cCells), a three-dimensional (3D) reduced graphene oxide (RGO) scaffold, by two approaches. In the first approach, the catalyst components were loaded on cells prior to carbonization (pre-loading method). Further pyrolysis produced NiFe oxides on RGO-like cCells. In the second approach, the cCells were synthesized first, followed by a hydrothermal reaction with the catalyst precursors (post-loading method). In comparison with the pre-loading method, the post-loading method enabled to load more nanocatalysts on individual cCells, which were highly efficient, with OER performance superior to that of the benchmark OER catalyst Ir/C.

In one aspect, the invention relates to a catalyst material for enhancing hydrogen and oxygen production, comprising algae-derived carbon scaffolds; and catalyst components coupled to the algae-derived carbon scaffolds.

In certain embodiments, the algae-derived carbon scaffolds comprise cCells.

In certain embodiments, the algae-derived carbon scaffolds are formed by carbonization of algae cells.

In certain embodiments, the algae cells comprise Tetraselmis cells, Nannochloropsis gaditana, Nannochloropsis oculate, or the likes.

In certain embodiments, the algae-derived carbon scaffolds comprise 3D RGO scaffolds.

In certain embodiments, the algae-derived carbon scaffolds comprise about 77 atomic % of C and about 14 atomic % of O.

In certain embodiments, the algae-derived carbon scaffolds contain C═C bonds, hydroxyl C—OH bonds, and ester C(═O)O bonds, wherein the C═C bonds are dominant bonds.

In certain embodiments, the catalyst components comprise OER and HER catalysts with earth-abundant materials, transition metal oxides/layer-double-hydroxides including NiFe oxide (NiFeOₓ), cobalt phosphate, perovskite oxides, and transition metal dichalcogenides including MoS₂.

In certain embodiments, the NiFe oxide has a molar ratio of Ni:Fe:O=6.7:6.1:26, with a formula of Ni_{1.1}FeO_{4.3}.

In certain embodiments, the catalyst material has a molar ratio of C:O:Ni:Fe≈49:35:6.7:6.1.

In certain embodiments, the catalyst material has a molar ratio of cCells to NiFe oxide, (C:O)_{cCell}:(Ni:Fe:O)_{NiFeOx}=49:9:6.7:6.1:26.

In certain embodiments, the catalyst material has about 39 wt. % of cCells and about 61 wt. % of NiFe oxide.

In certain embodiments, the catalyst material has Ni species mostly in the +2 oxidation state (NiOₓH_{y}) with N i2p_{3/2} binding energies close to 856 eV, and Fe species mostly in the +3 oxidation state (Fe₂O₃/FeOOH) with Fe 2p_{3/2} binding energies close to 711 eV.

In certain embodiments, the catalyst material has OER performance superior to that of a benchmark OER catalyst Ir/C.

In another aspect, the invention relates to an electrochemical device for hydrogen and oxygen production, comprising at least one electrode comprising the catalyst material as disclosed above.

In yet another aspect, the invention relates to a method for synthesizing a catalyst material for enhancing hydrogen and oxygen production, comprising filling algal cells with Ni²⁺ ions and Fe³⁺ ions to form a Ni²⁺/Fe³⁺@Cell composite comprising the Ni²⁺ and Fe³⁺ ions and the algal cells; mixing NH₃.H₂O with the Ni²⁺/Fe³⁺@Cell composite to form a NiFe(OH)ₓ@Cell composite comprising NiFe(OH)ₓ and the algal cells; mixing tetramethoxysilane (TMOS) with the NiFe(OH)ₓ@Cell composite to form a NiFe(OH)ₓ@Cell-SiO₂ composite comprising NiFe(OH), the algal cells and SiO₂; pyrolyzing the NiFe(OH)ₓ@Cell-SiO₂ composite at a temperature in a range of about 500-900° C. to form a NiFeOₓ@cCell-silica composite comprising NiFe(OH), cCell and silica; and removing the silica from the NiFeOₓ@cCell-silica composite to obtain the catalyst material.

In certain embodiments, said filling the algal cells with the Ni²⁺ ions and the Fe³⁺ ions to form the Ni²⁺/Fe³⁺@Cell composite comprises adding the algae cells into a first solution containing the Ni²⁺ ions and the Fe³⁺ ions to form a first mixture thereof, and shaking the first mixture for a period of time at room temperature, then centrifuging and washing the first mixture using DI water until the upper solution is colorless and no precipitates are formed when a NaOH solution is added, and collecting solids as the Ni²⁺/Fe³⁺@Cell composite.

In certain embodiments, the first solution has a mole ratio of Ni²⁺:Fe³⁺=3:1.

In certain embodiments, said mixing the NH₃.H₂O with the Ni²⁺/Fe³⁺@Cell composite to form the NiFe(OH)ₓ@Cell composite comprises mixing the Ni²⁺/Fe³⁺@Cell composite with a second solution containing DI water, ethanol and concentrated NH₃.H₂O to form a second mixture; and shaking the second mixture for a second period of time, then centrifuging and washing the second mixture until a final pH˜8.93 in the upper solution, and collecting solids as the NiFe(OH)ₓ@Cell composite.

In certain embodiments, said mixing TMOS with the NiFe(OH)ₓ@Cell composite to form the NiFe(OH)ₓ@Cell-SiO₂ composite comprises mixing the NiFe(OH)ₓ@Cell composite with a third solution containing DI water, ethanol and TMOS to form a third mixture; and shaking the third mixture to form a homogeneous gel and drying homogeneous gel to obtain the NiFeOₓ@Cell-SiO₂ composite.

In certain embodiments, said pyrolyzing is performed in N₂.

In certain embodiments, said removing the silica from the NiFeOₓ@cCell-silica composite comprises adding the NiFeOₓ@cCell-SiO₂ composite into a fourth solution containing NaOH to form a fourth mixture; heating the fourth mixture to a temperature in a range of about 60-120° C. on a hot plate and keeping the fourth mixture for about 4 hours at the temperature with mild stirring, and then cooling the fourth mixture down to room temperature; and centrifuging, washing with DI water, and dry the fourth mixture to obtain the NiFeOₓ@cCell.

In yet another aspect, the invention relates to method for synthesizing a catalyst material for enhancing hydrogen and oxygen production, comprising preparing a cell suspension comprising algal cells; mixing tetramethoxysilane (TMOS) with a cell suspension to form a Cell-SiO₂ composite; pyrolyzing the Cell-SiO₂ composite at a temperature in a range of about 500-900° C. to form a carbonized Cell-SiO₂ (cCell-SiO₂) composite; removing silica from the cCell-SiO₂ composite to obtain the carbonized cells (cCells); loading precursors into the cCells to form a precursor-cCell mixture; and performing hydrothermal reaction on the precursor-cCell mixture for 2-4 hours at a temperature in a range of about 100-250° C. to obtain the catalyst material.

In certain embodiments, the algal cells comprise tetraselmis cells.

In certain embodiments, said mixing TMOS with the cell suspension to form the Cell-SiO₂ composite comprises shaking a mixture of the TMOS with the cell suspension for one day at room temperature to obtain the Cell-SiO₂ composite.

In certain embodiments, said pyrolyzing is performed in N₂.

In certain embodiments, said removing silica from the cCell-SiO₂ composite comprises heating a mixture of the cCell-SiO₂ composite with a NaOH solution to a temperature in a range of about 60-120° C. for about 2-6 hours on a hot plate, and then cooling the mixture down to room temperature; and centrifuging, washing, and drying the mixture to obtain the carbonized cells (cCells).

In certain embodiments, the composition of the cCell comprises 77 atomic % of C and 14 atomic % of O.

In certain embodiments, said loading the precursors into the cCells comprises preparing a metal ion mixed solution containing Ni²⁺ and Fe³⁺ ions; and adding the cCells into the metal ion mixed solution to form the precursor-cCell mixture.

In certain embodiments, the metal ion mixed solution has a mole ratio of Ni²⁺:Fe³⁺=3:1, and the precursor-cCell mixture has a mole ratio of C:Ni²⁺:Fe³⁺=13:21:7.

In certain embodiments, the metal ion mixed solution has a pH of 5.88, the precursor-cCell mixture has a pH of 5.91, and after the hydrothermal reaction, the resulting mixture is centrifuged, and the pH of the upper solution is 5.87.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Carbon Nanostructures Made from Carbon-Neutral Algal Cells for Growth of NiFe Oxide Water-Splitting Nanocatalysts for Enhanced Hydrogen and Oxygen Production

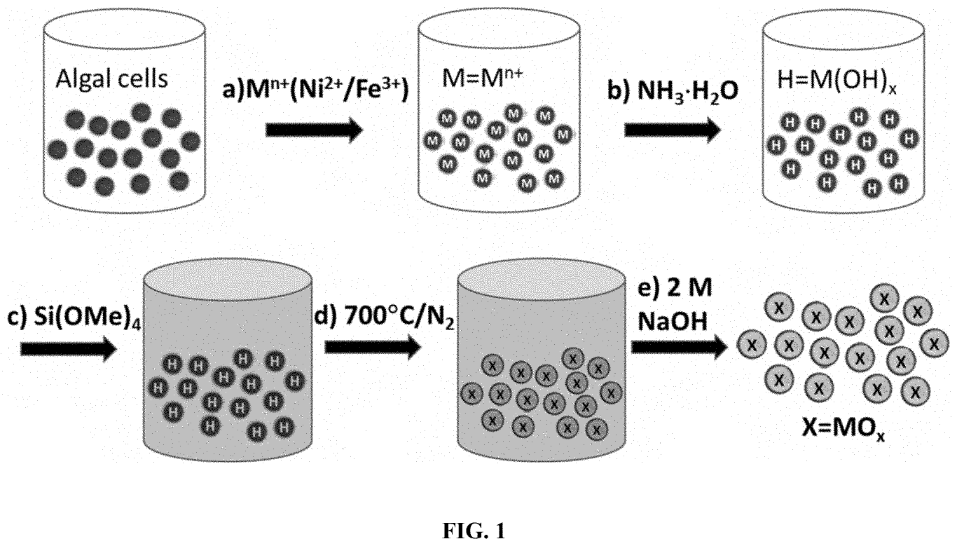

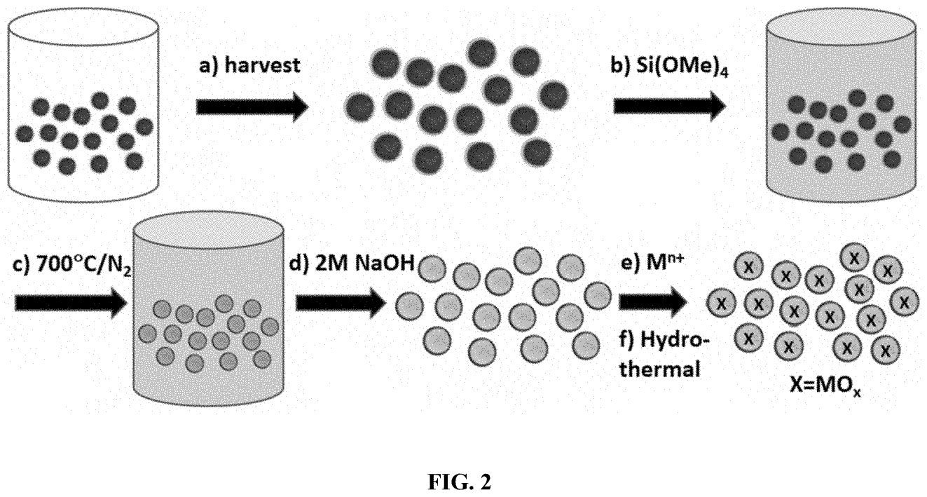

In this exemplary study, algae-derived carbon scaffolds were developed to grow electrolytic water-splitting NiFe oxide (NiFeOₓ) nanocatalysts, which is a highly promising class of oxygen evolution reaction (OER) catalysts. Specifically, Tetraselmis algal cells were selected as a model alga, and two approaches were explored to synthesize water-splitting nanocatalysts on Tetraselmis-derived carbon scaffolds. The first approach was called pre-loading method as schematically shown in FIG. 1, in which the catalyst components were loaded prior to cell carbonization. The second approach was called post-loading method as shown in FIG. 2, in which the catalyst components were loaded after carbonized cell (cCell) scaffolds were synthesized. It was found that the post-loading method produced highly efficient NiFeOₓ@cCell nanocatalysts, with more nanoparticles loading on individual cCells. The nanocatalysts presented excellent OER performance superior to that of the benchmark OER catalyst Ir/C.

### Methods and Characterization

Preparation of Algae Cells:

Tetraselmis algal cells (Florida Aqua Farms, Inc.) were grown at room temperature in artificial seawater (pH 8.15). Briefly, 25.08 g of sea salt were dissolved in 1 L deionized (DI) water (MilliQ water 18.2 MG cm). The pH of the seawater was adjusted to 8.15 using 0.5 M HCl solution and 0.5 M NaOH solution. The seawater was sterilized in a 900 W microwave oven for 8 min. After the seawater was cooled down to room temperature, 8 drops of Micro Algae Grow (Florida Aqua Farms, Inc.) per litter solution were added to the seawater, then algae cell seeds from a microalgae disk were added to the seawater. Air was bubbled through the culture, and 70 μmol photon m⁻² s⁻¹ of photosynthetically active radiation from cool white fluorescent light illuminated one side. The growth curve was determined by measuring OD₇₃₀ and cell counting with a hemocytometer. Cells in late logarithmic phase (˜8 days) were harvested and washed with DI water for further experiments.

Preparation of Cell-Templated Pre-Loading Catalysts:

The scheme used to synthesize pre-loading catalysts is shown in FIG. 1, which is described in detail as follows.

A first solution in 50 mL, containing 0.3 M Ni²⁺ ions and 0.1 M Fe³⁺ ions was prepared, according to the mole ratio [Ni²⁺]/[Fe³⁺]=3:1, from Ni(NO₃)₂.6H₂O and Fe(NO₃)₃.9H₂O.

Algae cells (˜4.5×10) were added into the above first solution. The mixture was shaken on a Daigger Vortex Genie 2 mixer for 40 hours at room temperature, then centrifuged and washed for at least three times using DI water, until the upper solution was colorless and no precipitates were formed when 0.5 M NaOH solution was added. The solid sample was collected and marked as Ni²⁺/Fe³⁺@Cell composite, as shown in step a) of FIG. 1.

5 ml of DI water, 5 ml of 95% ethanol, and 3 ml of concentrated NH₃.H₂O were mixed to form a second solution, then the Ni²⁺/Fe³⁺@Cell composite was mixed with the second solution. The mixture was shaken for 40 hours, then centrifuged and washed at least five times using DI water, until a final pH˜8.93 in the upper solution. The solid sample was marked as NiFe(OH)ₓ@Cell composite, as shown in step b) of FIG. 1.

5 ml of DI water, 5 ml of 95% ethanol, and 3 ml of tetramethoxysilane (TMOS), Si(OMe)₄ were mixed to form a third solution, then the NiFe(OH)ₓ@Cell composite was mixed with the third solution. The mixture was shaken on the Daigger Vortex Genie 2 mixer to form a homogeneous gel and dried overnight. Here the sample was marked as NiFeOₓ@Cell-SiO₂ composite, as shown in step c) of FIG. 1.

The NiFeOₓ@Cell-SiO₂ composite was calcinated at 700° C. for 3 hours in a tube furnace under the protection of N₂. The resulting sample was marked as NiFeOₓ@cCell-SiO₂ composite, where cCell stands for carbonized cells, as shown in step d) of FIG. 1.

About 0.5 g of NiFeOₓ@cCell-SiO₂ composite were added into 200 ml solution of 2 M NaOH solution. The mixture was heated to 90° C. on a hot plate and kept for 4 hours at this temperature with mild stirring. When the mixture was cooled down to room temperature, it was centrifuged, washed with DI water, and dried in a desiccator. The sample was marked as pre-loading NiFeOₓ@cCell, as shown in step e) of FIG. 1.

Preparation of Carbonized Cells and Post-Loading Catalysts:

The scheme used to synthesize post-loading catalysts is shown in FIG. 2. After harvesting the cells, as shown in step a) of FIG. 2, 3 mL of TMOS were mixed with 10 mL of cell suspension (˜2.5×10¹⁰ Tetraselmis cells). The mixture was shaken on the Daigger Vortex Genie 2 mixer for one day to get the Cell-SiO₂ composite at room temperature, as shown in step b) of FIG. 2. The Cell-SiO₂ composite was calcinated at 700° C. for 1 hour in the tube furnace under the protection of N₂ to obtain the carbonized cCell-SiO₂ composite, as shown in step c) of FIG. 2. 0.5 g of the carbonized cCell-SiO₂ composite were added into 200 mL NaOH aqueous solution (2 M). The mixture was heated to 90° C. for 4 hours on a hot plate. The mixture was then cooled down to room temperature, centrifuged, washed, and dried in a desiccator to get the carbonized cells (cCells), as shown in step d) of FIG. 2. The composition of the cCell sample, with 77% C and 14% 0, was determined by X-ray photoelectron spectroscopy (XPS).

10.5 mL of a mixed solution containing Ni²⁺ and Fe³⁺ ions (Ni²⁺:Fe³⁺=3:1) were prepared, using 7.0 mL of 0.3 M Ni²⁺ solution and 3.5 mL of 0.2 M Fe³⁺ solution, made from nickel (II) acetate tetrahydrate and ammonium iron (III) citrate. Then cCells in a calculated amount were added into the mixed solution, so that the mole ratio of C:Ni²⁺:Fe³⁺=13:21:7, as shown in step e) of FIG. 2, based on our previous work on reduced graphene oxide compositions. The mixture was transferred to a 23 mL Teflon-lined autoclave (Model #4749, Parr) and reacted for 3 hours at 180° C. The sample was washed with sufficient DI water to remove unreacted metal salts to get the post-loading NiFeOₓ@cCell, as shown in step f) of FIG. 2. The pH of the mixture before and after the reaction was monitored. It was 5.88 for the metal ion mixed solution. After adding cCells, the pH was changed to 5.91. After the hydrothermal reaction, the resulting mixture was centrifuged. The pH of the upper solution was 5.87.

Structure Characterization:

The samples were characterized by XPS, Raman spectroscopy, scanning electron microscopy (SEM), and transmission electron microscopy (TEM). XPS samples were drop-dried onto silicon substrates and measured on a K-Alpha X-ray XPS System equipped with monochromatic Al Kα (1486.6 eV). Raman spectroscopy was performed using an EZRaman-N microscope (excitation wavelength 532 nm) at 50% power, at room temperature, on solid samples drop-dried on silicon wafers. The morphology and microstructure of the samples were analyzed using a JEOL 7000F SEM. TEM imaging was performed using JEOL TEM Model 2010, operated at 200 kV, with energy-dispersive X-ray spectroscopy (EDS) analysis of the composition.

Preparation of Samples for Electrochemical Measurements:

Glassy carbon electrodes from CHI (3 mm in diameter, CHI104P) were polished and cleaned using the polishing kit (CHI120). Ni foam (1.6 mm thickness with a surface density of 346 g/m²) from MTI Co. was cut with a working area of 0.5×0.5 cm². Prior to use, the Ni foam was cleaned by sonication (2 min) in ethanol and DI water, respectively. 1 mg of the catalyst, 100 μL of DI water, 100 μL of ethanol, 5 μL of 5 wt % nafion solution (Sigma-Aldrich), and 0.5 mg graphite were mixed in a 1 mL microcentrifuge tube and the mixture was sonicated for ˜1 hour in an ice bath to get a homogeneous catalyst ink. Inks without adding graphite were also prepared for comparison and no significant difference was observed. Afterwards, 5 μl of the ink was drop-casted and dried on to a glassy carbon electrode of 3 mm in diameter (loading of about 0.35 mg/cm²). For Ni foam electrodes, the Ni foam electrode was weighed before dropping-cast. Then 50 μL of the homogeneous ink were drop-casted on the Ni foam and dried. Once the electrode was dried, the loading mass (1.2 mg/cm²) was determined by the weight change before and after dropping-cast. To prevent possible loss of the coated catalyst during the OER reaction, the Ni foam working area (0.5×0.5 cm²) was protected by sandwiching two pieces of bare Ni foams of the same area. Similarly, the OER benchmark standard, 20% Ir on Vulcan-XC-72 from Premetek Co. was prepared on glassy carbon electrodes and Ni foam electrodes using the same method. In addition, for pre-loading NiFeOₓ@cCell samples on Ni foams, the catalyst ink without involving sonication was also prepared to preserve the 3D structure of the cCells.

Electrochemical Measurements:

To evaluate the electrochemical OER catalytic activities, a standard three-electrode electrochemical system was investigated using a BASi Epsilon electrochemical workstation. The catalyst ink-loaded glassy carbon electrode or Ni foam electrode were used as a working electrode. A Pt wire electrode (CHI115) mounted in a CTFE cylinder was used as a counter electrode. A saturated calomel electrode (SCE, CHI150) was used as the reference electrode with a potential of 1.043 V versus RHE in 1 M KOH, calibrated against a HydroFlex hydrogen reference electrode (ET070, EQAD). The KOH solution was prepared from KOH pellets (certified ACS, Fischer Chemical) without further purification to remove possible iron impurity. It should be noted that when using Ni foams, the possible Fe impurity may enhance their OER activities by forming NiFe oxides, as observed by other groups. A three-electrode cell (CHI220) was used in the measurements. The electrochemistry workstation was used for the cyclic voltammetry (CV), the linear sweep voltammetry (LSV), and the chronopotentiometry (CP). The CV measurements were conducted in a voltage window from −0.8 to 0.8 V (vs SCE) with scan rates typically of 10-100 mV/s. The LSV measurements were performed in a potential window of 0-0.8V (vs SCE) under a constant sweep rate of 5 mV/s. The CP measurement in a three-electrode configuration (vs SCE) was conducted on a current density of 40 mA/cm² for a post-loading NiFeOₓ@cCell sample on Ni foam electrode of 0.5×0.5 cm² working area. The potentials shown in the main text were referred to RHE and were iR-corrected, unless noted. All electrochemical measurements were performed under 1 atmosphere in air and at room temperature.

### Results and Discussion

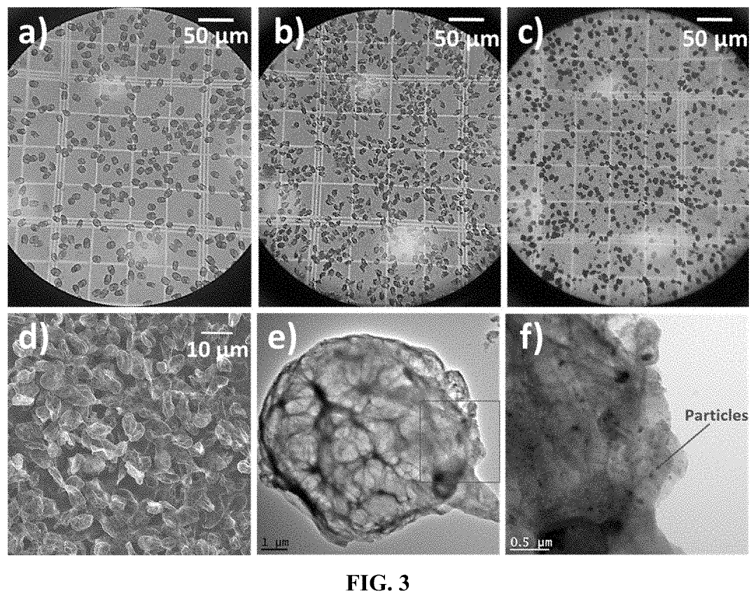

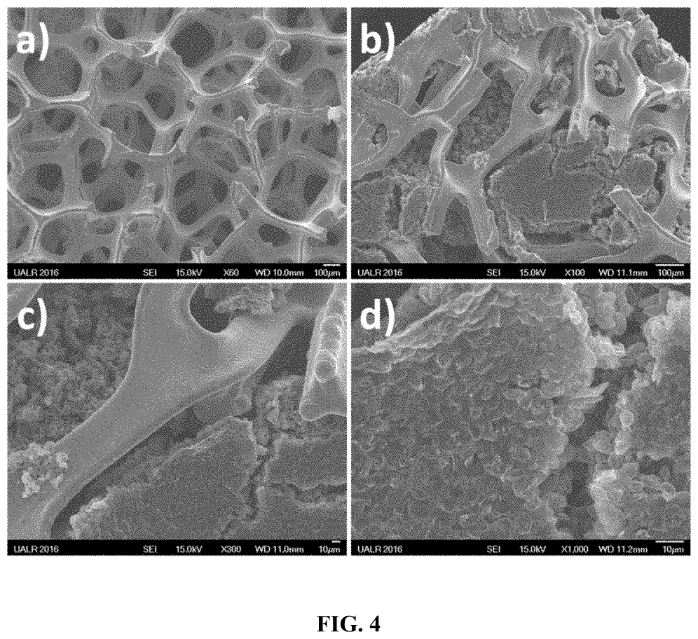

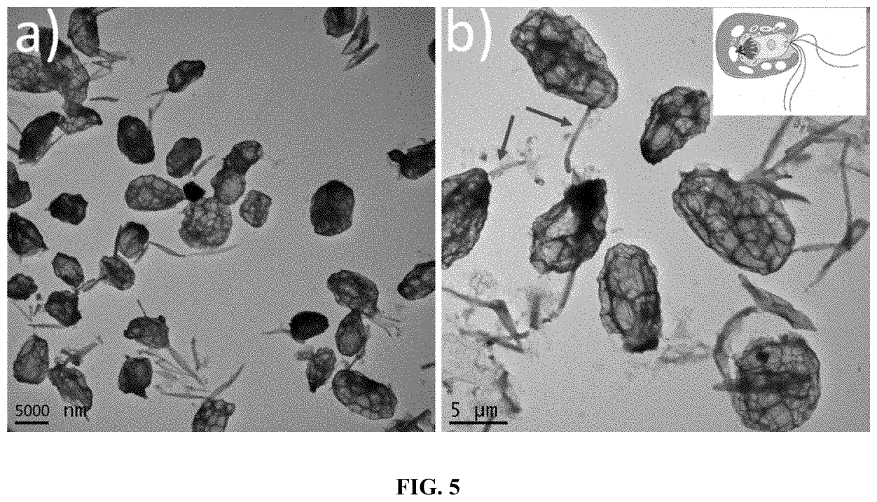

Tetraselmis cells used in this work are motile green, ovoid and slightly flattened, measured 9-15 μm×7-8 μm×4.5-6 μm, with 4 equal flagella. As shown in panel a) of FIG. 3, the pristine cell size is about 8 μm×14 μm. For the pre-loading method, the cell morphology was observed and shown in panels b)-c) of FIG. 3 after each treatment step from step a) to step e) of FIG. 1. Most of the resulting cCells presented as individual cells with 3D morphology after pyrolysis, shown by the SEM image in panel d) of FIG. 3 and FIG. 4, and the TEM images in panels e)-f) of FIG. 3 and FIGS. 5-7. Even the carbonized flagella of the cells were also observed as shown in FIG. 5, where the inset in FIG. 5 is a broad lateral view of a Tetraselmis cell with four flagella.

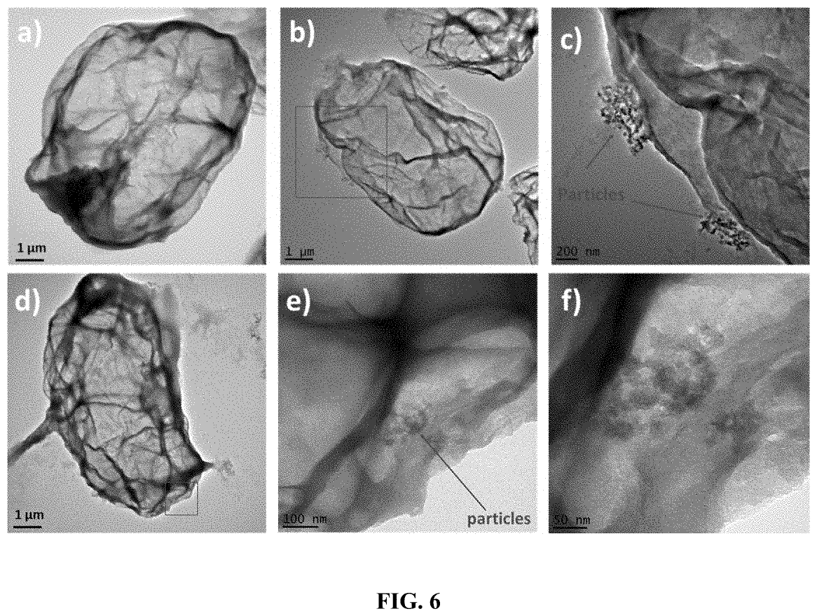

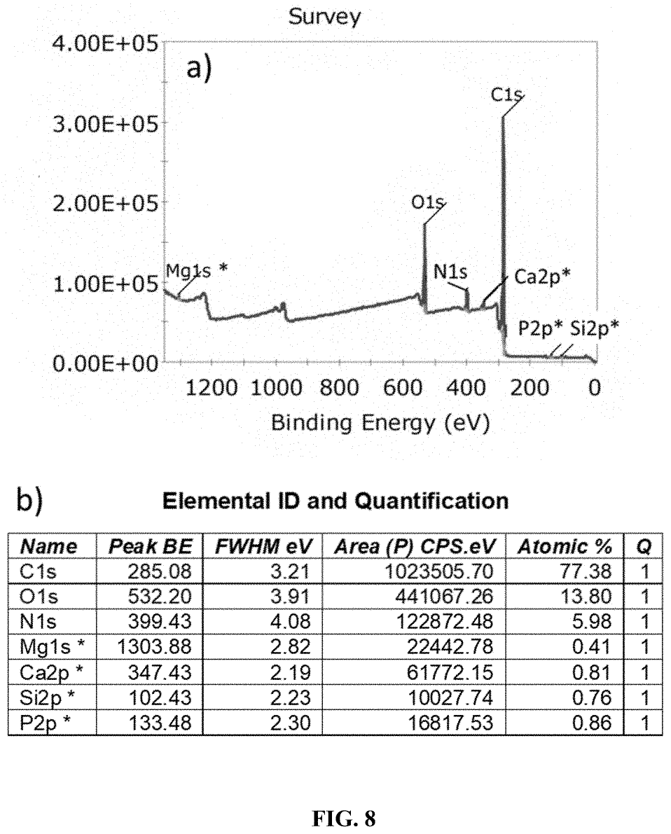

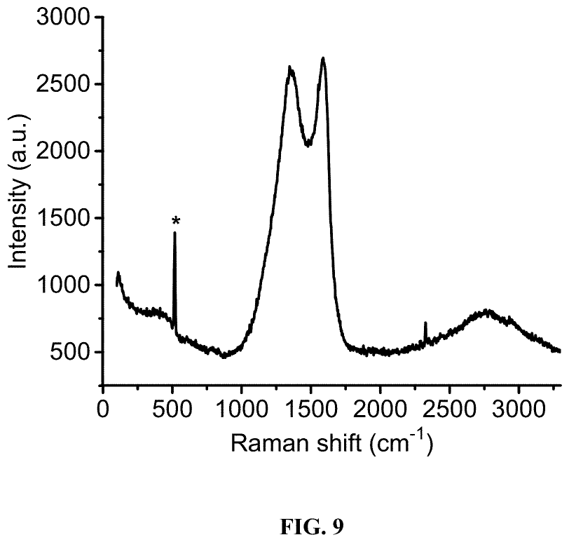

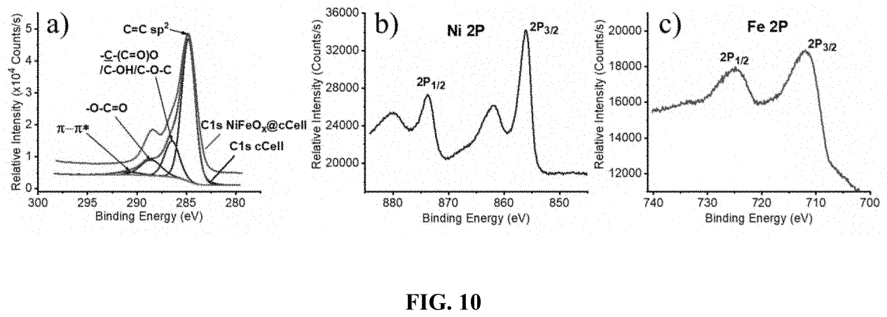

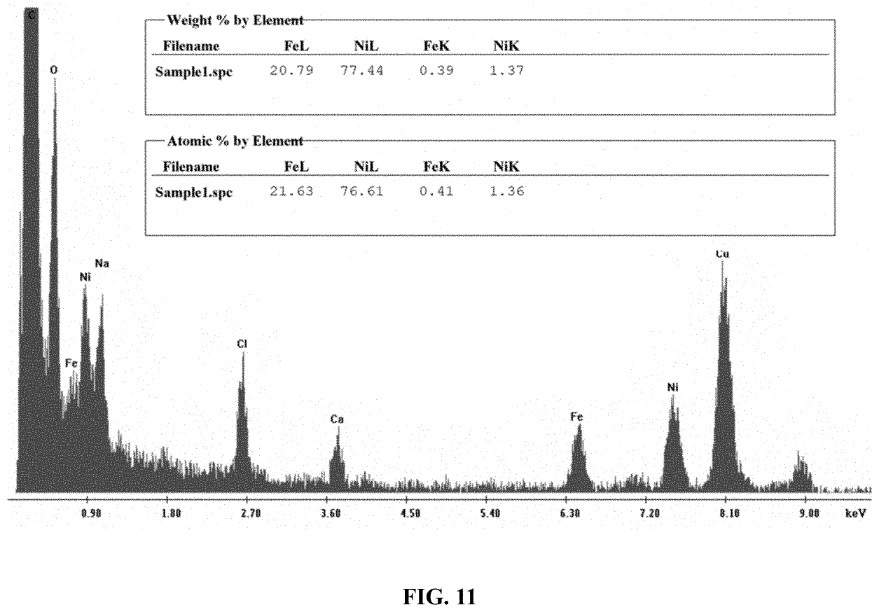

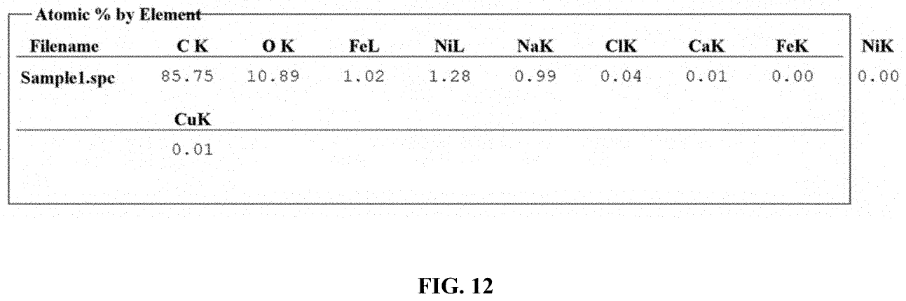

Based on the SEM and TEM images, a typical cCell size was about 6 μm×10 μm, a little shrinking from the pristine cells. The main elements in pure cCells are C and O, with a composition similar to reduced graphene oxide (RGO), containing about 77% C and 14% O, as estimated from XPS (FIG. 8). The Raman measurement shown in FIG. 9 also revealed the RGO-like structure of the cCell sample, with the D band at 1350 cm⁻¹ and the G band at 1590 cm⁻¹. These bands are characteristic of RGO. The result indicated that the after pyrolysis, the algae cells were turned into reduced graphene oxide. XPS shown in panel a) of FIG. 10 further confirmed the observation and was discussed in the next. As a pre-loading method for NiFeOₓ catalyst, it was confirmed that NiFeOₓ nanoparticles (<25 nm) were embedded in cCells (FIG. 6). EDS analysis suggested that for the NiFeOₓ@cCell sample, the ratio of [Ni]:[Fe] is close to 3.4:1 (FIG. 11), close to the starting stoichiometric ratio 3:1 used in the experiment. However, a careful inspection of TEM images revealed that only about 25% of the cCells were partially filled with the catalyst, forming NiFeOₓ@cCell composite, while the remaining was mainly pure cCells, as shown in panel a) of FIG. 6. A general composition survey using EDS analysis was conducted on the large areas of metal-ion containing individual cCells in a pre-loading NiFeOₓ@cCell composite sample (FIG. 12). It revealed that the total content of Ni and Fe ions was relatively low, about 10 wt % on the metal ion-containing cCells. Since only 25% cCells contained Ni and Fe ions, about 2.5 wt % of Ni and Fe ions were present in the composite sample. The low occupancy by NiFeOₓ catalyst in cCells might be responsible for the low OER current density observed in electrochemical measurements. To improve the catalyst loading in cCells, we further explored the post-loading method shown in FIG. 2.

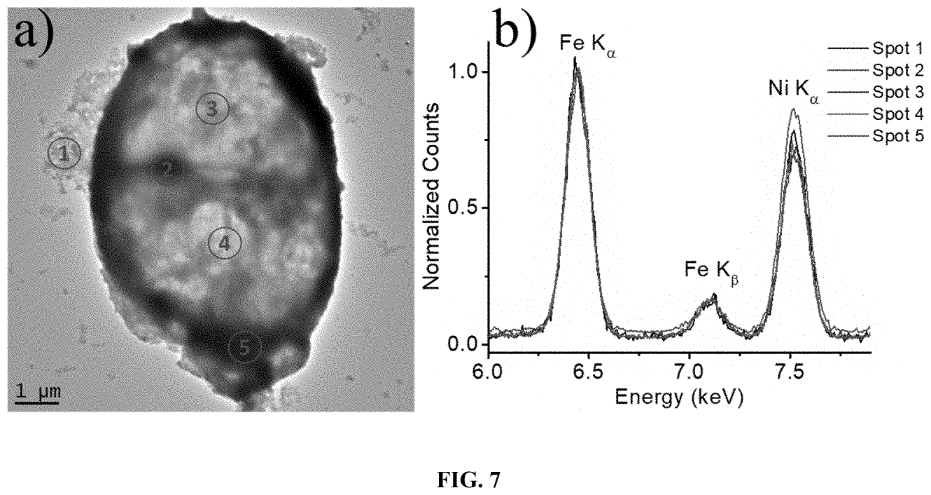

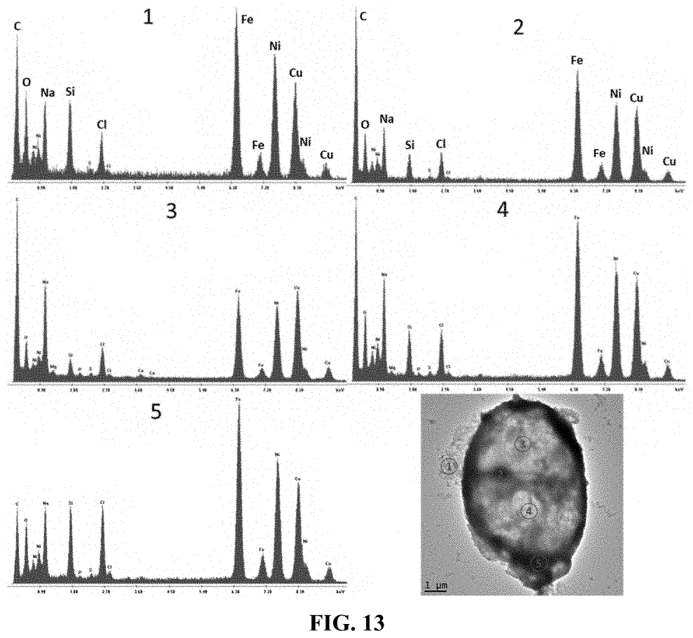

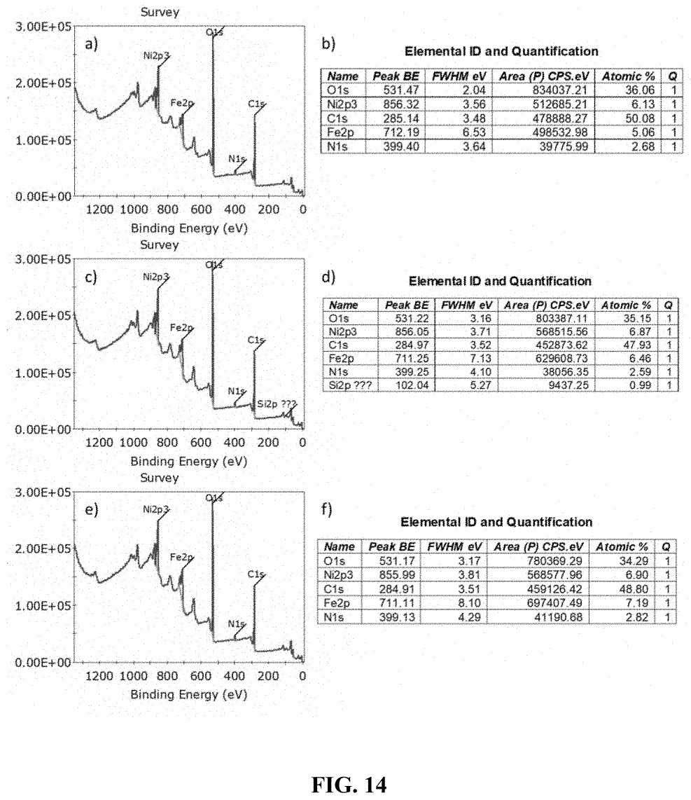

In the second approach, the cCells were synthesized first via the steps a) to d) shown in FIG. 2. Then the catalyst was loaded using the steps e) and f) shown in FIG. 2 via a hydrothermal reaction. As shown in the TEM image of a cCell, as shown in panel a) of FIG. 7, the as-prepared sample presented an entire loading of NiFeOₓ catalyst on the cCell, as indicated by the EDS spectra collected in 5 different spots on the cCell, as shown in panel b) of FIG. 7 and FIG. 13. The composition of the resulting NiFeOₓ@cCell composite was further analyzed by XPS as shown in panels b)-c) of FIG. 10 and FIG. 14.

C1s XPS analysis revealed that the cCells contained the dominant C═C bonds (˜284.8 eV), hydroxyl C—OH (˜286 eV), and ester C(═O)O (˜289 eV) bonds, as shown in panel a) of FIG. 10. The π-π* shake-up satellite peak was observed for the cCells around ˜290 eV. This indicated that the delocalized π conjugation, a characteristic of aromatic C structure, existed in cCells, similar to RGO foam samples. For the Cis XPS spectrum of the NiFeOₓ@cCell sample, as shown in panel a) of FIG. 10, in addition to the dominant C═C bonds (˜284.8 eV), the peaks of hydroxyl C—OH (˜286 eV) and ester C(═O)O (˜289 eV) bonds were also observed. XPS spectra shown in panels b)-c) of FIG. 10 also verified the existence of both Ni and Fe in the NiFeOₓ@cCell sample. From the Ni 2p spectra (FIG. 10, panel b), the Ni species was mostly in the +2 oxidation state (NiOₓH_{y}) with Ni 2p_{3/2} binding energies close to 856 eV. With Fe 2p_{3/2} binding energies close to 711 eV from the Fe 2p spectra (FIG. 10, panel c), the Fe species was mostly in the +3 oxidation state (Fe₂O₃/FeOOH).

For the chemical composition of the NiFe oxide-loaded samples, the average atomic percentage of C:O:Ni:Fe was approximately 49:35:6.7:6.1, as shown in the XPS spectra with Ar ion sputtering (FIG. 14). Since we knew that carbon is from the cCells and the oxygen content is the sum of the oxygen containing in both the cCells and the NiFe oxide, we are able to estimate the Ni:Fe:O ratio by subtracting the oxygen from cCells which has an atomic percentage of C:O equal to 77:14 (FIG. 8). With 49 atomic % C, about 9 atomic % O are from the cCells. Thus, we can estimate that in NiFe oxide-loaded sample, among 35 atomic % O, 26 atomic % O are from NiFe oxide by subtracting the 9 atomic % from the 35 atomic %. The resulting Ni:Fe:O ratio is equal to 6.7:6.1:26, with a formula of Ni_{1.1}FeO_{4.3} for the NiFe oxide loaded on the cCells. The molar ratio of cCells to NiFe oxide can be found as (C:O)_{cCell}:(Ni:Fe:O)_{NiFeOx}=49:9:6.7:6.1:26. From this ratio, the weight % of cCells and NiFe oxide was estimated to be 39% and 61%, respectively. As noted, the Ni:Fe ratio was deviated from the starting ratio of 3:1, suggesting there is a portion of Ni ions not involving in the reaction. The cause is under further investigation.

In our previous studies on the porous 3D structures of RGO foam, the functional groups, mainly located on GO sheets edges, such as hydroxyl, carboxyl, and epoxy groups, were covalently interconnected and cross-linked with each other during the hydrothermal process, thereby forming a monolithic 3D chemically linked RGO network. This unique 3D structure can accommodate the active sites of NiFe oxide nanoparticles, facilitate their electron transfer at electrode surfaces, and maintain their electrochemical activities. While for the NiFeOₓ@cCell sample synthesized in this work, each individual cCell served as a micro 3D RGO scaffold where NiFe oxide nanoparticles were grown on.

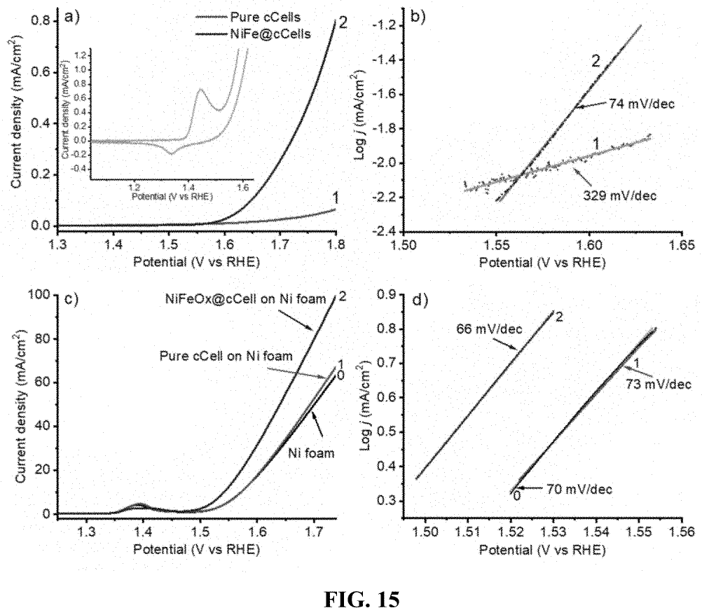

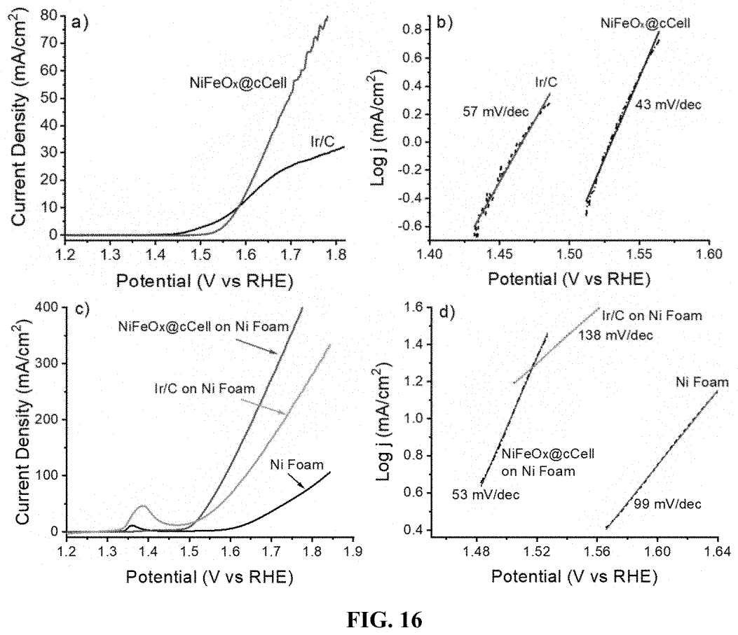

Electrochemical measurements were performed to evaluate the OER performance of the pre-loading NiFeOₓ@cCell samples and the post-loading NiFeOₓ@cCell samples, based on the onset potential, the overpotential at 10 mA/cm², and the Tafel slope. With units in mV/decade (mV/dec), the Tafel slope determines the additional voltage required to increase the catalytic current by an order of magnitude. The cyclic voltammogram (CV) of a pre-loading NiFeOₓ@cCell sample on a glassy carbon electrode is shown in the insert of panel a) of FIG. 15, where a scan rate of 25 mV/s was applied. The peak at 1.44 V vs RHE is assigned to the Ni(II)/Ni(III or IV)redox process. The polarization curves of the pre-loading NiFeOₓ@cCell sample are shown in panel a) of FIG. 15, where the peak at 1.44 V was suppressed due to the lower scan rate of 5 mV/s. It is known that the peak current increased linearly with the square root of the scan rate by the Randles-Sevcik equation. The polarization curves of the pre-loading NiFeOₓ@cCell sample presented much better OER performance than the pure cCell sample. However, as compared with those of NiFeOₓ on RGO samples and that of the post-loading NiFeOₓ@cCell sample in panel a) of FIG. 16, the OER current density of the pre-loading NiFeOₓ@cCell sample was much weaker, less than 1 mA/cm² even at a potential of 1.8 V vs RHE. This low current density could be caused by few active catalytic sites due to the low occupancy of NiFeOₓ catalyst on cCells, as observed from the TEM measurements (FIG. 6).

Interestingly, when depositing the pre-loading NiFeOₓ@cCell sample on Ni foam electrodes, an enhancement of the OER current density was observed as shown in panel c) of FIG. 15, with an enhancement factor of 1.6 at 1.7 V. In comparison, the pure cCell sample on Ni foam almost had the same OER activity as the Ni foam, indicating a negligible impact. As observed from previous studies, the addition of a small amount of iron (III) ions to NiOₓHₓ catalysts formed NiFe oxides, which enhanced the OER activity significantly. The result observed here was consistent with these findings, where as an active OER catalyst, the NiFeOₓ from the pre-loading NiFeOₓ@cCell sample may be responsible for the enhancement.

In addition, it is known that the possible Fe impurity from a KOH solution may make the OER activity of Ni foam stronger by forming NiFe oxides. However, the OER activity of the synthesized sample is stronger than that of the Ni foam, as shown in panel c) of FIG. 15. Therefore, the possible Fe impurity in the base KOH solution will not alter the findings in this work. On the other hand, possible Fe impurity shows no apparent effects on glassy carbon electrodes, as observed in panel a) of FIG. 15, where negligible OER activity of pure cCells on the glassy carbon electrode was observed.

The polarization curves were fitted to the Tafel equation η=b log (j/j₀), where η is the overpotential, b is the Tafel slope, j is the current density, and j₀ is the exchange current density. The Tafel slopes and the slope values, were displayed in panel b) of FIG. 15 for the samples on glassy carbon electrodes and in panel d) of FIG. 15 for the samples on Ni foam electrodes. In panel b) of FIG. 15, the pure cCells had a high Tafel slope of 329 mV/dec. In a sharp contrast, the Tafel slope of the pre-loading NiFeOₓ@cCell was reduced significantly to 74 mV/dec, a dramatic improvement of OER performance. For the NiFe oxide sample on Ni foam electrode, less improvement was observed. Its Tafel slope (66 mV/dec) was slightly lower than that of pure Ni foam electrode (70 mV/dec).

For the post-loading NiFeOₓ@cCell sample on a glassy carbon electrode, which contained more NiFe oxide nanoparticles on the cCells, the polarization curves in panel a) of FIG. 16 clearly show that the catalytic sample was able to highly efficiently enhance oxygen evolution reaction as an electrocatalyst. It achieved a current density of 58 mA/cm² at 1.7 V, while the benchmark OER catalyst Ir/C sample only achieved 26.6 mA/cm² at the same potential. The observed results were reproducible by repeatedly measuring the polarization curves of the post-loading NiFeOₓ@cCell sample and Ir/C sample prepared from different batches. The result suggested that the post-loading NiFeOₓ@cCell sample had a much better OER electrocatalytic ability than Ir/C. In addition, the sample achieved a current density of 10 mA/cm² at the potential of ˜1.58 V, the same potential as that of Ir/C. Its onset of oxygen evolution took place at ˜1.50 V, a little bit higher than 1.43 V of Ir/C, as listed in Table 1.

TABLE 1
Comparison of OER properties of NiFe oxide and
Ir/C electrocatalysts in 1M KOH solution.

| | Onset | Potential at | Tafel | |
|---|---|---|---|---|
| | potential | 10 mA/cm² | slope | Refer- |
| Samples | (V) | (V) | (mV/dec) | ences |
| NiFeOₓ@cCell | 1.49 | >>1.8 | 73 | This |
| (pre-loading) | | | | invention |
| NiFeOₓ@cCell | 1.50 | 1.58 | 43 | This |
| (post-loading) | | | | invention |
| NiFeOₓ@cCell | 1.46 | 1.50 | 53 | This |
| (post-loading) on | | | | invention |
| Ni Foam | | | | |
| Ir/C | 1.43 | 1.58 | 57 | This |
| | | | | invention |
| Ir/C on Ni Foam | 1.47 | ~1.48 | 138 | This |
| | | | | invention |
| RGO-Ni—Fe Foam | 1.46 | 1.62 | 57 | Ref. [14] |
| Ni—Fe-CNT | 1.45 | 1.47 | 31 | Ref. [12] |
| Pristine Ni—Fe-CFP | 1.50 | 1.57 | 44.0 | Ref. [13] |
| 2-cycle Ni—Fe-CFP | 1.43 | 1.48 | 31.5 | Ref. [13] |
| Ir/C | 1.47 | 1.52 | 39.2 | Ref. [13] |

The Tafel slopes of the post-loading NiFeOₓ@cCell sample and Ir/C sample were shown in panel b) of FIG. 16, along with the fitting curves and the slope values. The post-loading NiFeOₓ@cCell sample exhibited a Tafel slope of 43 mV/dec in 1 M KOH. This value was better than that of the Ir/C reference (57 mV/dec) and close to the literature value ˜40 mV/dec. It is noted that no iR compensation was applied in the measurements. The Tafel slopes could be smaller, if the iR compensation would be applied, being more closer to those in the listed references. The result suggested that the post-loading NiFeOₓ@cCell sample had excellent OER performance superior to that of benchmark OER catalyst Ir/C. The difference in OER current densities between the post-loading NiFeOₓ@cCell sample and Ir/C sample at a given OER potential, for example, at 1.7 V vs RHE in panel a) of FIG. 16, could be caused by the difference in a few factors, in addition to the intrinsic properties of the catalysts. These factors include the number of active sites, the conductivity of the scaffolds, and the surface area needed for electron transfer and ion transport in the samples. The result suggests that the post-loading NiFeOₓ@cCell sample was better over these factors than Ir/C, and could be further improved by optimizing these factors.

In agreement with the pre-loading NiFeOₓ@cCell on Ni foam, when loaded on Ni foams, the post-loading NiFeOₓ@cCell sample enhanced OER current density significantly, about 7.6 times larger than the Ni foam and 1.7 times larger than Ir/C on Ni foam at 1.7 V vs RHE, as shown in panel c) of FIG. 16. The onset of oxygen evolution of the post-loading NiFeOₓ@cCell sample took place at 1.46 V, better than 1.47 V of Ir/C (Table 1). In addition, the sample achieved a current density of 10 mA/cm² at the potential of 1.50 V, close to the literature values of 1.47 V and 1.48 V. The potential of Ir/C at the current density of 10 mA/cm² was estimated to be ˜1.48 V, whose accurate position was interfered by the tail of the oxidation peak at 1.38 V.

The post-loading NiFeOₓ@cCell sample on Ni foam exhibited a small value of Tafel slope of 53 mV/dec, as shown in panel d) of FIG. 16, while the Tafel slope of Ir/C on Ni foam was 138 mV/dec, even larger than the Tafel slope of pure Ni foam (99 mV/dec, without iR compensation). This might be partly caused by the interference of the tail of the oxidation peak at 1.38 V, for which, further study is needed. Together with a few benchmark NiFe oxide electrocatalysts from other research groups, the electrochemical performances of the pre-loading and the post-loading NiFeOₓ@cCell samples were summarized in Table 1. The OER properties of the post-loading NiFeOₓ@cCell sample on Ni foam are close to those of 2-cycle Ni—Fe—CFP and comparable with those of other listed superior samples, demonstrating promising potential.

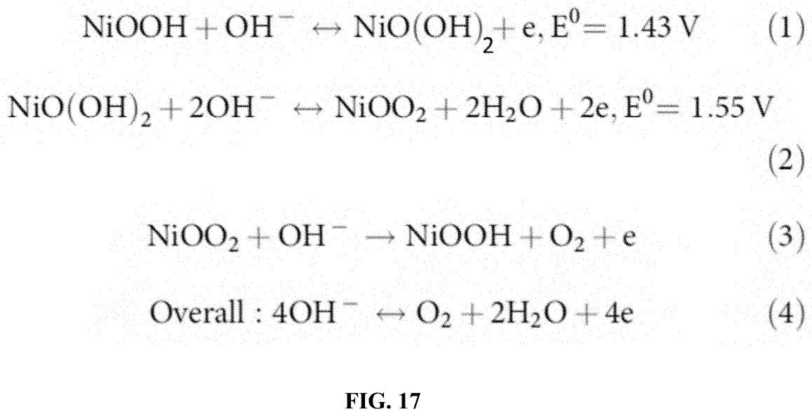

The observed outstanding OER performance of the post-loading samples with lower Tafel slopes suggested some changes in the kinetics of the overall reaction and could be better understood by examining the intermediate steps in OER reaction mechanisms (FIG. 17). As shown in FIG. 17, among the steps, step (1) and (2) are reversible reactions. Step (3) is irreversible and is the rate-determining step for the overall rate of the process. As discussed by Zhao group, catalysts are mostly used to facilitate the kinetics of step (3). The NiOOH species serves as active centers in the OER reaction, promoting the oxidation of OH⁻ into molecular oxygen. The enhancing role of Fe played in Ni-based OER catalysts could be related to introducing additional edge/defect structures, which could be further investigated in the future experiments.

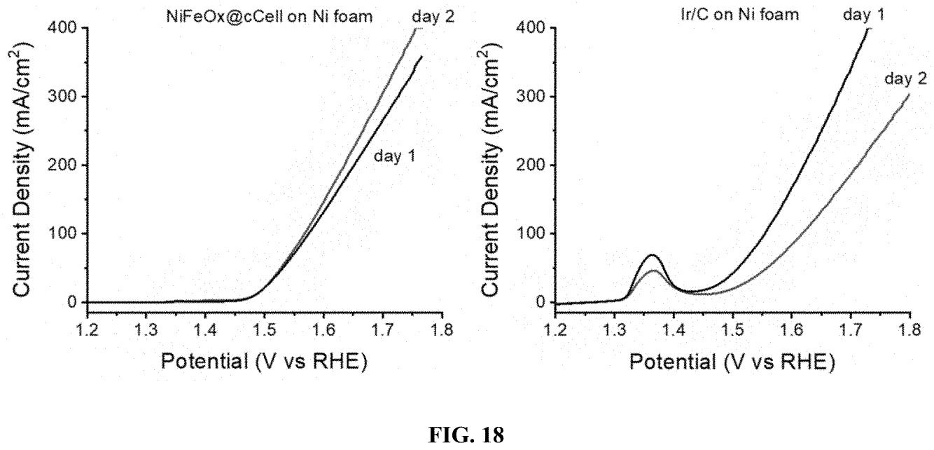

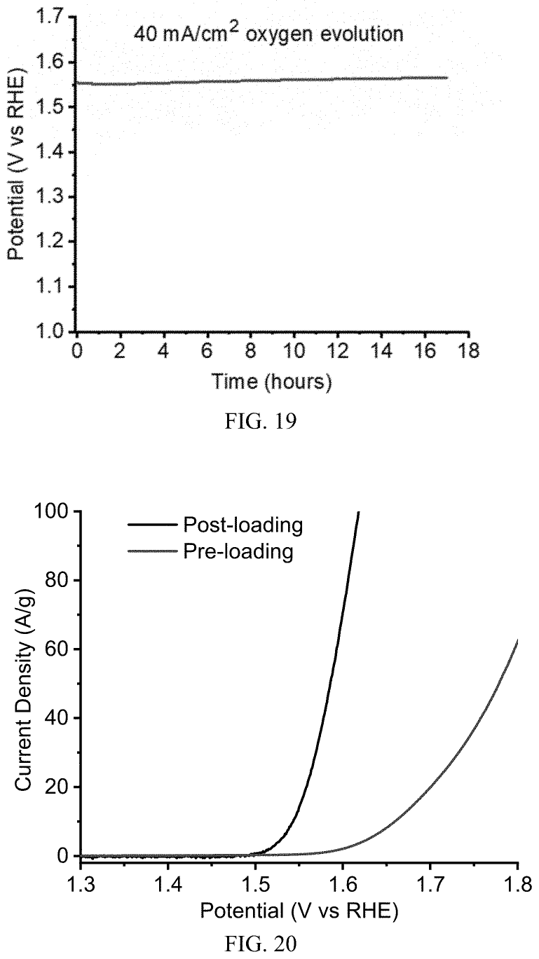

Furthermore, the post-loading NiFeOₓ@cCell sample also presented impressive stability in 1 M KOH, as compared with Ir/C on Ni foam. As shown in panel a) of FIG. 18, no degradation in OER activity was observed for the post-loading NiFeOₓ@cCell sample after one day measurement. In contrast, a significant decay in OER current density was observed for Ir/C on Ni foam, as shown in panel b) of FIG. 18, consistent with the previous report. Further chronopotentiometric measurement of the post-loading NiFeOₓ@cCell sample on Ni foam at 40 mA/cm² (FIG. 19) revealed a constant potential at 1.56 V vs RHE over 17 hours, confirming the stability of the catalytic sample. The observed stability of the post-loading NiFeOₓ@cCell sample agreed with those of the carbon-supported NiFe oxide catalysts. As discussed previously, the improved stability could be related to reactive oxygen species (ROS) scavenging properties of graphene-based materials. ROS generated in the water oxidation progress contribute to the instability of catalytic materials. These graphene-based materials present self-recovery capability from oxidation in alkaline conditions. They are able to scavenge reactive oxygen species to enhance the catalyst stability. Since cCells are RGO-like, containing similar oxygen functional groups to oxidized CNTs, and have favorable electron mobility and unique surface properties, they may serve as an efficient scaffold by accommodating the active species and facilitate their electron transfer at electrode surfaces, as demonstrated in this work.

As discussed early, the low OER activity of the pre-loading samples might be mainly due to few active catalytic sites caused by low occupancy of the catalyst on cCells where about 2.5 wt % of Ni and Fe ions were present in the sample. Assuming the apparent formula with 4 oxygen per iron, as determined from XPS data in FIG. 14, we estimated that about 3.7 wt % of NiFeOₓ were present in the pre-loading sample. In comparison, the NiFeOₓ content in the post-loading sample was much higher, 61 wt % as calculated early. With the information, we were able to compare the pre-loading sample's OER activity per NiFeOₓ mass with that of the post-loading sample by plotting the current density in A/g NiFeOₓ as shown in FIG. 20. We found that at 10 A/g NiFeOₓ, the overpotential of the post-loading sample is 1.54 V, lower than 1.66 V of the pre-loading sample. The observed overpotential 1.54 V at 10 A/g for the post-loading sample was close to those of RuO₂ and IrO₂ summarized by Dai's group. The result further supported that the OER activity of the post-loading sample is better than that of the pre-loading sample. Additionally, the OER activity could also be related to the effective surface area, which can be determined by double-layer capacitance measurements or Brunauer-Emmett-Teller (BET) surface area analysis, which will be discussed elsewhere.

When reviewing the reaction route of the pre-loading method, there are three main limitations that may prevent this method from wide adoption. For the first limitation, the metal ion loading steps a)-b) in FIG. 1 could be important in determining how much metal ions will be adsorbed onto the cells. The algal cells contain a variety of functional sites including carboxyl, imidazole, sulfydryl, amino, phosphate, sulfate, thioether, phenol, carbonyl, amide, and hydroxyl moieties that could be responsible for metal adsorption. The cell wall includes polysaccharides, proteins, and lipids with charged functional groups such as carboxyl, hydroxyl and amine groups. From the observed low occupancy result, it is noted that the binding of metal ions to the algal cells may not be strong and the metal ions could be easily washed away during the wash step. In addition, even after metal oxide catalyst was loaded on cCells, caution should also be paid to sample wash due to peptization, which may cause loss of product as well. For the second limitation, the high temperature annealing may cause the possible growth of larger nanoparticles. And for the third limitation, the NaOH treatment to dissolve SiO₂ might also inadequately remove some of the NiFe oxide nanoparticles. With these limitations on the pre-loading method, future work will focus on the study of the post-loading method. In addition to investigate the facile routes to synthesize cCells without using SiO₂ template tetramethoxysilane, various factors in the hydrothermal reactions will also be evaluated systematically, including the compositions of C:Ni:Fe, temperatures, pH levels, and solvents. For example, the optimal atomic composition is 25% Fe and 75% Ni found in literature. The composition obtained in this work for the post-loading sample is about 48% Fe and 52% Ni. Although the variation of the OER activity may be only within a factor of 2 or less when the iron content changed from 25% to 50%, further composition optimization is still possible by adjusting the composition to 25% Fe. With these modifications, the OER performance of the post-loading NiFeOₓ@cCell samples could have a room for further improvement.

### CONCLUSIONS

In the exemplary example, Tetraselmis algal cells were used as a model template for the first time to synthesize OER nanocatalysts NiFe oxides on Tetraselmis-derived cCells to form a 3D micro, reduced graphene oxide-like scaffold. Two approaches were explored, the pre-loading method and the post-loading method. The pre-loading method did not yield highly efficient OER nanocatalysts, due to the low occupancy of the NiFeOₓ nanocatalyst on cCells, limited by the reaction route. In comparison, the post-loading method produced highly efficient NiFeOₓ@cCell nanocatalysts with OER performance superior to that of the benchmark OER catalyst Ir/C, which offers great potential for using carbon-neutral algae-based products.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Pruvost, J.; Comet, J. F.; Borgne, F. L.; Goetz, V.; Legrand, J. Theoretical Investigation of Microalgae Culture in the Light Changing Conditions of Solar Photobioreactor Production and Comparison with Cyanobacteria. Algal Research 2015, 10, 87-99.
[2]. Ritter, S. K. Climate Change Award: Algenol. C&E News 2015, 93, 34-35.
[3]. Ma, X.-N.; Chen, T.-P.; Yang, B.; Liu, J.; Chen, F. Lipid Production from Nannochloropsis. Mar. Drugs 2016, 14, 61.
[4]. Umdu, E. S.; Tuncer, M.; Seker, E. Transesterification of Nannochloropsis oculata Microalga's Lipid to Biodiesel on Al₂O₃ Supported CaO and MgO Catalysts. Bioresour. Technol. 2009, 100, 2828-2831.
[5]. Zhao, E. H.; Watanabe, F.; Zhao, W. Nonlinear Optical Transmission of Cyanobacteria-Derived Optical Materials. Opt. Mat. 2015, 46, 497-503.
[6]. Gong, M.; Dai, H. J. A Mini Review of NiFe-Based Materials as Highly Active Oxygen Evolution Reaction Electrocatalysts. Nano Res. 2015, 8, 23-39.
[7]. Trotochaud, L.; Young, S. L.; Ranney, J. K.; Boettcher, S. W. Nickel-Iron Oxyhydroxide Oxygen-Evolution Electrocatalysts: The Role of Intentional and Incidental Iron Incorporation. J. Am. Chem. Soc. 2014, 136, 6744-6753.
[8]. Huang, J.; Han, J.; Wang, R.; Zhang, Y.; Wang, X.; Zhang, X.; Zhang, Z.; Zhang, Y.; Song, B.; Jin, S. Improving Electrocatalysts for Oxygen Evolution Using NiₓFe₃₋ₓO₄/Ni Hybrid Nanostructures Formed by Solvothermal Synthesis. ACS Energy Lett. 2018, 3, 1698-1707.
[9]. Louie, M. W.; Bell, A. T. An Investigation of Thin-Film Ni—Fe Oxide catalysts for the Electrochemical Evolution of Oxygen. J. Am. Chem. Soc. 2013, 135, 12329-37.
[10]. Song, F.; Bai, L.; Moysiadou, A.; Lee, S.; Hu, C.; Liardet, L.; Hu, X. Transition Metal Oxides as Electrocatalysts for the Oxygen Evolution Reaction in Alkaline Solutions: An Application-Inspired Renaissance. J. Am. Chem. Soc. 2018, 140, 7748-7759.
[11]. Zhou, H.; Yu, F.; Zhu, Q.; Sun, J.; Qin, F.; Luo, Y.; Bao, J.; Yu, Y.; Chen, S.; Ren, Z. Water Splitting by Electrolysis at High Current Density under 1.6 Volt. Energy Environ. Sci. 2018, DOI: 10.1039/C8EE00927A.
[12]. Gong, M.; Li, Y.; Wang, H.; Liang, Y.; Wu, J. Z.; Zhou, J.; Wang, J.; Regier, T.; Wei, F.; Dai, H. An Advanced Ni—Fe Layered Double Hydroxide Electrocatalyst for Water Oxidation. J. Am. Chem. Soc. 2013, 135, 8452-8455.
[13]. Wang, H.; Lee, H.-W.; Deng, Y.; Lu, Z.; Hsu, P.-C.; Liu, Y.; Lin, D.; Cui, Y. Bifunctional Non-Noble Metal Oxide Nanoparticle Electrocatalysts through Lithium-Induced Conversion for Overall Water Splitting. Nat. Commun. 2015, 6, 7261.
[14]. Wang, D. Y.; Watanabe, F.; Zhao, W. One-Pot Growth of 3D Reduced Graphene Oxide Foams Embedded with NiFe Oxide Nanocatalysts for Oxygen Evolution Reaction. J. Electrochem. Soc. 2016, 163, F3158-F3163.
[15]. Gong, M.; Zhou, W.; Tsai, M.-C.; Zhou, J.; Guan, M.; Lin, M.-C.; Zhang, B.; Hu, Y.; Wang, D.-Y.; Yang, J.; Pennycook, S. J.; Hwang, B.-J.; Dai, H. J. Nanoscale Nickel Oxide/Nickel Heterostructures for Active Hydrogen Evolution Electrocatalysis. Nat. Commun. 2014, 5, 4695.
[16]. Li, Y.; Wang, H.; Xie, L.; Liang, Y.; Hong, G.; Dai, H. MoS₂ Nanoparticles Grown on Graphene: An Advanced Catalyst for the Hydrogen Evolution Reaction. J. Am. Chem. Soc. 2011, 133, 7296-7299.
[17]. Miao, J.; Xiao, F.-X.; Yang, H. B.; Khoo, S. Y.; Chen, J.; Fan, Z.; Hsu, Y.-Y.; Chen, H. M.; Zhang, H.; Liu, B. Hierarchical Ni—Mo—S Nanosheets on Carbon Fiber Cloth: A Flexible Electrode for Efficient Hydrogen Generation in Neutral Electrolyte. Sci. Adv. 2015, 1, e1500259.
[18]. Li, Y.; Kuttiyiel, K. A.; Wu, L.; Zhu, Y.; Fujita, E.; Adzic, R. R.; Sasaki, K. Enhancing Electrocatalytic Performance of Bifunctional Cobalt-Manganese-Oxynitride Nanocatalysts on Graphene. ChemSusChem 2017, 10, 68-73.
[19]. Courchesne, N.-M. D.; III, S. A. S.; Cantú, V. J.; Hammond, P. T.; Belcher, A. M. Biotemplated Silica and Silicon Materials as Building Blocks for Micro- to Nanostructures. Chem. Mater. 2015, 27, 5361-5370.
[20]. Courchesne, N.-M. D.; Klug, M. T.; Chen, P.-Y.; Kooi, S. E.; Yun, D. S.; Hong, N.; Fang, N. X.; Belcher, A. M.; Hammond, P. T. Assembly of a Bacteriophage-Based Template for the Organization of Materials into Nanoporous Networks. Adv. Mater. 2014, 26, 3398-3404.
[21]. Nuraje, N.; Dang, X.; Qi, J.; Allen, M. A.; Lei, Y.; Belcher, A. M. Biotemplated Synthesis of Perovskite Nanomaterials for Solar Energy Conversion. Adv. Mater. 2012, 24, 2885-2889.
[22]. Giese, M.; Blusch, L. K.; Khan, M. K.; MacLachlan, M. J. Functional Materials from Cellulose-Derived Liquid-Crystal Templates. Angew. Chem.-Int. Edit. 2015, 54, 2888-2910.
[23]. Shopsowitz, K. E.; Hamad, W. Y.; MacLachlan, M. J. Chiral Nematic Mesoporous Carbon Derived from Nanocrystalline Cellulose. Angew. Chem.-Int. Edit. 2011, 50, 10991-10995.
[24]. Xia, Y.; Xiao, Z.; Dou, X.; Huang, H.; Lu, X.; Yan, R.; Gan, Y.; Zhu, W.; Tu, J.; Zhang, W.; Tao, X. Green and Facile Fabrication of Hollow Porous MnO/C Microspheres from Microalgaes for Lithium-Ion Batteries. ACS Nano 2013, 7, 7083-7092.
[25]. He, J.; Zi, G.; Yan, Z.; Li, Y.; Xie, J.; Duan, D.; Chen, Y.; Wang, J. Biogenic C-Doped Titania Templated by Cyanobacteria for Visible-Light Photocatalytic Degradation of Rhodamine B. J. Enviro. Sci. 2014, 26, 1195-1202.
[26]. Zhang, B.; Yang, S.; Zhang, Y.; Wang, Q.; Ren, T. Biotemplate-Directed Fabrication of Size-Controlled Monodisperse Magnetic Silica Microspheres. Colloids Surf B Biointerfaces 2015, 131, 129-135.
[27]. Bi, L.; Pan, G. Facile and Green Fabrication of Multiple Magnetite Nano-cores @Void@Porous Shell Microspheres for Delivery Vehicles. J. Mater. Chem. A 2014, 2, 3715-3718.
[28]. Tao, X.; Wu, R.; Xia, Y.; Huang, H.; Chai, W.; Feng, T.; Gan, Y.; Zhang, W. Biotemplated Fabrication of Sn@C Anode Materials Based on the Unique Metal Biosorption Behavior of Microalgae. ACS Appl. Mater. Interfaces. 2014, 6, 3696-3702.
[29]. Hoek, C. v. d.; Mann, D. G.; Jahns, H. M. Algae: An Introduction to Phycology. Cambridge University Press: New York, 1995.
[30]. Hoff, F. H.; Snell, T. W. Plankton Culture Manual. 6th ed.; Florida Aqua Farms, Inc.: Dade City, Fla., 2004.
[31]. Tang, C.; Wang, H. S.; Wang, H. F.; Zhang, Q.; Tian, G. L.; Nie, J. Q.; Wei, F. Spatially Confined Hybridization of Nanometer-Sized NiFe Hydroxides into Nitrogen-Doped Graphene Frameworks Leading to Superior Oxygen Evolution Reactivity. Adv. Mater. 2015, 27, 4516-4522.
[32]. Scholz, M. J.; Weiss, T. L.; Jinkerson, R. E.; Jing, J.; Roth, R.; Goodenough, U.; Posewitz, M. C.; Gerken, H. G. Ultrastructure and Composition of the Nannochloropsis gaditana Cell Wall. Eukaryotic Cell 2014, 13, 1450-1464.
[33]. Friebel, D.; Louie, M. W.; Bajdich, M.; Sanwald, K. E.; Cai, Y.; Wise, A. M.; Cheng, M. J.; Sokaras, D.; Weng, T. C.; Alonso-Mori, R.; Davis, R. C.; Bargar, J. R.; Norskov, J. K.; Nilsson, A.; Bell, A. T. Identification of Highly Active Fe Sites in (Ni,Fe)OOH for Electrocatalytic Water Splitting. J. Am. Chem. Soc. 2015, 137, 1305-1313.
[34]. Stevens, M. B.; Trang, C. D. M.; Enman, L. J.; Deng, J.; Boettcher, S. W. Reactive Fe-Sites in Ni/Fe (Oxy)hydroxide Are Responsible for Exceptional Oxygen Electrocatalysis Activity. J. Am. Chem. Soc. 2017, 139, 11361-11364.
[35]. González, M. A.; Aguayo, P. A.; Inostroza, I. d. L.; Castro, P. A.; Fuentes, G. A.; Gomez, P. I. Ultrastructural and Molecular Characterization of Tetraselmis Strains (Chlorodendrophyceae, Chlorophyta) Isolated from Chile. Gayana Bot. 2015, 72, 47-57.
[36]. Arora, M.; Anil, A. C.; Leliaert, F.; Delany, J.; Mesbahi, E. Tetraselmis indica (Chlorodendrophyceae, Chlorophyta), A New Species Isolated from Salt Pans in Goa, India. Eur. J. Phycol. 2013, 48, 61-78.
[37]. Perrozzi, F.; Prezioso, S.; Ottaviano, L. Graphene oxide: from fundamentals to applications. J. Phys.: Condens. Matter 2015, 27, 013002.
[38]. Bagri, A.; Mattevi, C.; Acik, M.; Chabal, Y. J.; Chhowalla, M.; Shenoy, V. B. Structural evolution during the reduction of chemically derived graphene oxide. Nat. Chem. 2010, 2, 581-587.
[39]. Mattevi, C.; Eda, G.; Agnoli, S.; Miller, S.; Mkhoyan, K. A.; Celik, O.; Mastrogiovanni, D.; Granozzi, G.; Garfunkel, E.; Chhowalla, M. Evolution of Electrical, Chemical, and Structural Properties of Transparent and Conducting Chemically Derived Graphene Thin Films. Adv. Funct. Mater. 2009, 19, 2577-2583.
[40]. Wang, D. Y.; Watanabe, F.; Zhao, W. Reduced Graphene Oxide-NiO Nanomembranes as Oxygen Evolution Reaction Electrocatalysts. ECS J. Solid State Sci. Technol. 2017, 6, M3049-M3054.
[41]. Hassel, B. A. v.; Burggraaf, A. J. Oxidation State of Fe and Ti Ions Implanted in Yttria-Stabilized Zirconia Studied by XPS. Appl. Phys. A 1991, 52, 410-417.
[42]. Wu, Y.; Yi, N.; Huang, L.; Zhang, T.; Fang, S.; Chang, H.; Li, N.; Oh, J.; Lee, J. A.; Kozlov, M.; Chipara, A. C.; Terrones, H.; Xiao, P.; Long, G.; Huang, Y.; Zhang, F.; Zhang, L.; Lepro, X.; Haines, C.; Lima, M. D.; Lopez, N. P.; Rajukumar, L. P.; Elias, A. L.; Feng, S.; Kim, S. J.; Narayanan, N. T.; Ajayan, P. M.; Terrones, M.; Aliev, A.; Chu, P.; Zhang, Z.; Baughman, R. H.; Chen, Y. Three-Dimensionally Bonded Spongy Graphene Material with Super Compressive Elasticity and Near-Zero Poisson's Ratio. Nat. Commun. 2015, 6, 6141.
[43]. Xu, Y.; Sheng, K.; Li, C.; Shi, G. Self-Assembled Graphene Hydrogel via a One-Step Hydrothermal Process. ACS Nano 2010, 4, 4324-4330.
[44]. Benck, J. D.; Hellstern, T. R.; Kibsgaard, J.; Chakthranont, P.; Jaramillo, T. F. Catalyzing the Hydrogen Evolution Reaction (HER) with Molybdenum Sulfide Nanomaterials. ACS Catal. 2014, 4, 3957-3971.
[45]. Lim, C. S.; Chua, C. K.; Sofer, Z.; Klimova, K.; Boothroyd, C.; Pumera, M. Layered Transition Metal Oxyhydroxides as Tri-Functional Electrocatalysts. J. Mater. Chem. A 2015, 3, 11920-11929.
[46]. Qiu, Y.; Xin, L.; Li, W. Electrocatalytic Oxygen Evolution over Supported Small Amorphous Ni—Fe Nanoparticles in Alkaline Electrolyte. Langmuir 2014, 30, 7893-7901.
[47]. Anson, F. C. Application of Potentiostatic Current Integration to the Study of the Adsorption of Cobalt (III)-(Ethylenedinitrilo(tetraacetate) on Mercury Electrodes. Anal. Chem. 1964, 36, 932-934.
[48]. Bard, A. J.; Faulkner, L. R. Electrochemical Methods: Fundamentals and Applications. 2^{nd} ed.; John Wiley & Sons: New York, 2001.
[49]. Burke, L. D. Oxide Growth and Oxygen Evolution on Noble Metals. In Electrodes of Conductive Metallic Oxides, Trasatti, S., Ed. Elsevier Scientific New York, 1980; Vol. Part A, pp 152-158.
[50]. Duan, J.; Chen, S.; Zhao, C. Ultrathin Metal-Organic Framework Array for Efficient Electrocatalytic Water Splitting. Nat. Commun. 2017, 8, 15341.
[51]. Qiu, Y.; Wang, Z.; Owens, A. C. E.; Kulaots, I.; Chen, Y.; Kane, A. B.; Hurt, R. H. Antioxidant Chemistry of Graphene-Based Materials and Its Role in Oxidation Protection Technology. Nanoscale 2014, 6, 11744-11755.
[52]. Park, H. S.; Leonard, K. C.; Bard, A. J. Surface Interrogation Scanning Electrochemical Microscopy (SI-SECM) of Photoelectrochemistry at a W/Mo—BiVO₄ Semiconductor Electrode: Quantification of Hydroxyl Radicals during Water Oxidation. J. Phys. Chem. C 2013, 117, 12093-12102.
[53]. Song, C. H.; Pehrsson, P. E.; Zhao, W. Recoverable Solution Reaction of HiPco Carbon Nanotubes with Hydrogen Peroxide. J. Phys. Chem. B 2005, 109, 21634-21639.
[54]. Tu, X. M.; Pehrsson, P. E.; Zhao, W. Redox Reaction of DNA-Encased HiPco Carbon Nanotubes with Hydrogen Peroxide: A Near Infrared Optical Sensitivity and Kinetics Study. J. Phys. Chem. C 2007, 111, 17227-17231.
[55]. Xu, Y.; Pehrsson, P. E.; Chen, L. W.; Zhang, R.; Zhao, W. Double-Stranded DNA Single-Walled Carbon Nanotube Hybrids for Optical Hydrogen Peroxide and Glucose Sensing. J. Phys. Chem. C 2007, 111, 8638-8643.
[56]. Dukovic, G.; White, B. E.; Zhou, Z.-Y.; Wang, F.; Jockusch, S.; Steigerwald, M. L.; Heinz, T. F.; Friesner, R. A.; Turro, N. J.; Brus, L. E. Reversible Surface Oxidation and Efficient Luminescence Quenching in Semiconductor Single-Wall Carbon Nanotubes. J. Am. Chem. Soc. 2004, 126, 15269-15276.
[57]. Benedict, B.; Pehrsson, P. E.; Zhao, W. Optically Sensing Additional Sonication Effects on HiPco Nanotubes in Aerated Water. J. Phys. Chem. B 2005, 109, 7778-7780.
[58]. Xu, Y.; Pehrsson, P. E.; Chen, L. W.; Zhao, W. Controllable Redox Reaction of Chemically Purified DNA-Single Walled Carbon Nanotube Hybrids with Hydrogen Peroxide. J. Am. Chem. Soc. 2008, 130, 10054-10055.
[59]. Zhao, E. H.; Ergul, B.; Zhao, W. Caffeine's Antioxidant Potency Optically Sensed with Double-Stranded DNA-Encased Single-Walled Carbon Nanotubes. J. Phys. Chem. B 2015, 119, 4068-4075.
[60]. Trotochaud, L.; Ranney, J. K.; Williams, K. N.; Boettcher, S. W. Solution-cast metal oxide thin film electrocatalysts for oxygen evolution. J. Am. Chem. Soc. 2012, 134, 17253-61.
[61]. Yeh, T.-F.; Syu, J.-M.; Cheng, C.; Chang, T.-H.; Teng, H. Graphite Oxide as a Photocatalyst for Hydrogen Production from Water. Adv. Funct. Mater. 2010, 20, 2255-2262.
[62]. Harris, D. C. Exploring Chemical Analysis. 5th ed.; W. H. Freeman: New York, N.Y., 2013.

What is claimed is:

1. A catalyst material for enhancing hydrogen and oxygen production, comprising:
algae-derived carbon scaffolds; and
catalyst components coupled to the algae-derived carbon scaffolds.

2. The catalyst material of claim 1, wherein the algae-derived carbon scaffolds comprise algae-derived carbonized cells (cCells).

3. The catalyst material of claim 2, wherein the algae-derived carbon scaffolds are formed by carbonization of algae cells.

4. The catalyst material of claim 3, wherein the algae cells comprise Tetraselmis cells, Nannochloropsis gaditana, Nannochloropsis oculate, or the likes.

5. The catalyst material of claim 2, wherein the algae-derived carbon scaffolds comprise three-dimensional (3D) reduced graphene oxide (RGO) scaffolds.

6. The catalyst material of claim 2, wherein the algae-derived carbon scaffolds comprise about 77 atomic % of C and about 14 atomic % of O.

7. The catalyst material of claim 2, wherein the algae-derived carbon scaffolds contain C═C bonds, hydroxyl C—OH bonds, and ester C(═O)O bonds, wherein the C═C bonds are dominant bonds.

8. The catalyst material of claim 2, wherein the catalyst components comprise OER and HER catalysts with earth-abundant materials, transition metal oxides/layer-double-hydroxides including NiFe oxide (NiFeOₓ), cobalt phosphate, perovskite oxides, and transition metal dichalcogenides including MoS₂.

9. The catalyst material of claim 8, wherein the NiFe oxide has a molar ratio of Ni:Fe:O=6.7:6.1:26, with a formula of Ni_{1.1}FeO_{4.3}.

10. The catalyst material of claim 9, wherein the catalyst material has a molar ratio of C:O:Ni:Fe≈49:35:6.7:6.1.

11. The catalyst material of claim 9, wherein the catalyst material has a molar ratio of cCells to NiFe oxide, (C:O)_{cCell}:(Ni:Fe:O)_{NiFeOx}=49:9:6.7:6.1:26.

12. The catalyst material of claim 9, wherein the catalyst material has about 39 wt. % of cCells and about 61 wt. % of NiFe oxide.

13. The catalyst material of claim 8, wherein the catalyst material has Ni species mostly in the +2 oxidation state (NiOₓH_{y}) with Ni 2p_{3/2} binding energies close to 856 eV, and Fe species mostly in the +3 oxidation state (Fe₂O₃/FeOOH) with Fe 2p_{3/2} binding energies close to 711 eV.

14. The catalyst material of claim 8, wherein the catalyst material has oxygen evolution reaction (OER) performance superior to that of a benchmark OER catalyst Ir/C.

15. An electrochemical device for hydrogen and oxygen production, comprising:
at least one electrode comprising the catalyst material of claim 1.

16. A method for synthesizing a catalyst material for enhancing hydrogen and oxygen production, comprising:
filling algal cells with Ni²⁺ ions and Fe³⁺ ions to form a Ni²⁺/Fe³⁺@Cell composite comprising the Ni²⁺ and Fe³⁺ ions and the algal cells;
mixing NH₃.H₂O with the Ni²⁺/Fe³⁺@Cell composite to form a NiFe(OH)ₓ@Cell composite comprising NiFe(OH)ₓ and the algal cells;
mixing tetramethoxysilane (TMOS) with the NiFe(OH)ₓ@Cell composite to form a NiFe(OH)ₓ@Cell-SiO₂ composite comprising NiFe(OH), the algal cells and SiO₂;
pyrolyzing the NiFe(OH)ₓ@Cell-SiO₂ composite at a temperature in a range of about 500-900° C. to form a NiFeOₓ@cCell-silica composite comprising NiFe(OH)ₓ, algae-derived carbonized cells (cCell) and silica; and
removing the silica from the NiFeOₓ@cCell-silica composite to obtain the catalyst material.

17. The method of claim 16, wherein said filling the algal cells with the Ni²⁺ ions and the Fe³⁺ ions to form the Ni²⁺/Fe³⁺@Cell composite comprises:
adding the algae cells into a first solution containing the Ni²⁺ ions and the Fe³⁺ ions to form a first mixture thereof, and
shaking the first mixture for a period of time at room temperature, then centrifuging and washing the first mixture using DI water until the upper solution is colorless and no precipitates are formed when a NaOH solution is added, and collecting solids as the Ni²⁺/Fe³⁺@Cell composite.

18. The method of claim 17, wherein the first solution has a mole ratio of Ni²⁺:Fe³⁺=3:1.

19. The method of claim 16, wherein said mixing the NH₃.H₂O with the Ni²⁺/Fe³⁺@Cell composite to form the NiFe(OH)ₓ@Cell composite comprises:
mixing the Ni²⁺/Fe³⁺@Cell composite with a second solution containing DI water, ethanol and concentrated NH₃.H₂O to form a second mixture; and
shaking the second mixture for a second period of time, then centrifuging and washing the second mixture until a final pH˜8.93 in the upper solution, and collecting solids as the NiFe(OH)ₓ@Cell composite.

20. The method of claim 16, wherein said mixing TMOS with the NiFe(OH)ₓ@Cell composite to form the NiFe(OH)ₓ@Cell-SiO₂ composite comprises:
mixing the NiFe(OH)ₓ@Cell composite with a third solution containing DI water, ethanol and TMOS to form a third mixture; and
shaking the third mixture to form a homogeneous gel and drying homogeneous gel to obtain the NiFeOₓ@Cell-SiO₂ composite.

21. The method of claim 16, wherein said pyrolyzing is performed in N₂.

22. The method of claim 16, wherein said removing the silica from the NiFeOₓ@cCell-silica composite comprises:
adding the NiFeOₓ@cCell-SiO₂ composite into a fourth solution containing NaOH to form a fourth mixture;
heating the fourth mixture to a temperature in a range of about 60-120° C. on a hot plate and keeping the fourth mixture for about 4 hours at the temperature with mild stirring, and then cooling the fourth mixture down to room temperature; and
centrifuging, washing with DI water, and dry the fourth mixture to obtain the NiFeOₓ@cCell.

23. A method for synthesizing a catalyst material for enhancing hydrogen and oxygen production, comprising:
preparing a cell suspension comprising algal cells;
mixing tetramethoxysilane (TMOS) with a cell suspension to form a Cell-SiO₂ composite;
pyrolyzing the Cell-SiO₂ composite at a temperature in a range of about 500-900° C. to form a carbonized Cell-SiO₂ (cCell-SiO₂) composite;
removing silica from the cCell-SiO₂ composite to obtain the carbonized cells (cCells);
loading precursors into the cCells to form a precursor-cCell mixture; and
performing hydrothermal reaction on the precursor-cCell mixture for 2-4 hours at a temperature in a range of about 100-250° C. to obtain the catalyst material.

24. The method of claim 23, wherein the algal cells comprise tetraselmis cells.

25. The method of claim 23, wherein said mixing TMOS with the cell suspension to form the Cell-SiO₂ composite comprises shaking a mixture of the TMOS with the cell suspension for one day at room temperature to obtain the Cell-SiO₂ composite.

26. The method of claim 23, wherein said pyrolyzing is performed in N₂.

27. The method of claim 23, wherein said removing silica from the cCell-SiO₂ composite comprises:
heating a mixture of the cCell-SiO₂ composite with a NaOH solution to a temperature in a range of about 60-120° C. for about 2-6 hours on a hot plate, and then cooling the mixture down to room temperature; and
centrifuging, washing, and drying the mixture to obtain the carbonized cells (cCells).

28. The method of claim 27, wherein the composition of the cCell comprises 77 atomic % of C and 14 atomic % of O.

29. The method of claim 23, wherein said loading the precursors into the cCells comprises:
preparing a metal ion mixed solution containing Ni²⁺ and Fe³⁺ ions; and
adding the cCells into the metal ion mixed solution to form the precursor-cCell mixture.

30. The method of claim 29, wherein the metal ion mixed solution has a mole ratio of Ni²⁺:Fe³⁺=3:1, and the precursor-cCell mixture has a mole ratio of C:Ni²⁺:Fe³⁺=13:21:7.

31. The method of claim 29, wherein the metal ion mixed solution has a pH of 5.88, wherein the precursor-cCell mixture has a pH of 5.91, and wherein after the hydrothermal reaction, the resulting mixture is centrifuged, and the pH of the upper solution is 5.87.
